(19) United States
(12) Patent Application Publication
(10) Pub. No.: US 2026/0193321 A1
(43) Pub. Date: Jul. 9, 2026

(54) MODIFIED GELATINS, HYDROGELS, AND PROCESSES FOR THEIR PRODUCTION

(71) Applicant: GELOMICS PTY LTD, Brisbane (AU)

(72) Inventors: Christoph Adalbert MEINERT, Brisbane (AU); Peter Andrew LEVETT, Brisbane (AU)

(73) Assignee: GELOMICS PTY LTD, Brisbane, QL (AU)

(21) Appl. No.: 19/133,596

(22) PCT Filed: Dec. 1, 2023
(86) PCT No.: PCT/AU2023/051242

§ 371 Date: May 28, 2025

(30) Foreign Application Priority Data
Dec. 2, 2022 (AU) 2022903674

(51) Int. Cl. C07K 14/78; C08J 3/075; C08J 3/24; C12N 5/00

(52) U.S. Cl. CPC C07K 14/78; C08J 3/075; C08J 3/24; C12N 5/0068; C08J 2389/06; C12N 2533/54; C12N 2537/10; C12N 2539/00

(57) ABSTRACT

Provided herein are modified gelatins, crosslinked modified gelatins and hydrogels, and processes for their production. Also provided herein are uses of such materials as matrices for cell growth.

This patent/patent application claims priority from Australian provisional patent application no. 2022903674, filed on 2 Dec. 2022, the entire contents of which are incorporated herein by this reference.

### TECHNICAL FIELD

The present disclosure relates to modified gelatins, crosslinked modified gelatins and hydrogels, and processes for their production. The present disclosure also relates to the use of such materials as matrices for cell growth. The present disclosure also relates to the use of such materials in bioprinting.

### BACKGROUND ART

Hydrogels are three-dimensional networks of hydrophilic polymers which have wide-ranging applications across numerous fields of endeavour. As many hydrogels are biocompatible, one particular field of use is biomedicine including in applications such as medical dressings, tissue engineering scaffolds, as super-absorbent materials and drug-delivery systems.

Gelatins are partially hydrolyzed collagen derivatives which can form hydrogels, and have a range of potential applications in biomedicine, having been proposed for use in matrices for cell growth, and 3D bioprinting. Gelatins can be obtained from a number of different sources including, for example, bovine and porcine sources, and a number of derivatives are also known.

However, commonly used mammalian gelatins and derivatives thereof display unfavorable rheological properties in solution including thermal, non-covalent, and reversible crosslinking at temperatures below their melting point at approximately 30° C., and high viscosity over 30° C. (see e.g. Van Den Bulcke et al.). Furthermore, the mechanical and biochemical properties of some crosslinked gelatin derivatives can be significantly impacted by the temperature at which the derivate is crosslinked (Loessner et al.). From a practical perspective, these properties render the necessity of heating and maintaining gelatin and derivative solutions at constant temperatures over 30° C. to enable liquid handling using manual and automated pipetting methods, and facilitate predictable, reproducible, and controllable physicochemical properties of crosslinked formulations. Furthermore, high viscosity of solutions over 30° C. adversely affect pipetting accuracy and repeatability, as well as the ability to be 3D printed using various printing methods.

As a result, handling and crosslinking of some gelatin and gelatin derivative solutions is difficult to control in an accurate, predictable, and reproducible manner, and these difficulties increase with the concentration of the gelatin or gelatin derivative. Furthermore, these properties limit such gelatin and gelatin derivative solutions in their ability to be handled accurately using automated liquid handling technology used in pharmaceutical drug discovery and development, as well as other laboratory processes.

Approaches for modifying gelatins include chemical derivatization with photocrosslinkable moieties, which enable radical polymerization in the presence of a photoinitiator and an appropriate light source. However, light-initiated crosslinking requires specialised equipment and additional reagents to facilitate crosslinking which add complexity and increase costs. Such photocrosslinking processes and devices are also difficult to integrate into applications including, but not limited to, automated high throughput drug discovery and inkjet, multijet or polyjet 3D printing.

It would be beneficial to provide further hydrogels useful as matrices for cell growth and/or 3D bioprinting applications, which can be conveniently used over a range of operating conditions, and which can be readily produced from starting materials without the need for specialised equipment.

### SUMMARY

The present inventors have identified that modified gelatin polymers derived from natural sources of cold-water adapted marine species, such as but not limited to salmon skin gelatin, with introduced chemical functional groups, such as thiol, amino, maleimide or vinyl sulfone, serve as new covalently crosslinkable biomaterials which can produce hydrogels with advantageous properties for the biomedical and food industries. Solutions of the resulting gelatin derivatives exhibit low viscosity, low melting temperatures, and can be readily crosslinked via click-chemistry reactions with crosslinker molecules containing appropriate reactive moieties. Example materials according to the present disclosure have been found to have low and substantially temperature-independent viscosity properties over a wide range of temperatures, as well as low melting points, and crosslinking times that can be tuned to be comparatively short or long as required for the intended application, depending on the reactive moiety in the crosslinker molecule. This enables solutions of the materials to be handled readily using manual and automated pipetting methods, as well as various 3D printing methods. For manual pipetting methods it may be beneficial to have a longer crosslinking time, for example it may enable preparation of a larger master volume which does not need to be used immediately. For 3D printing applications, in some embodiments a shorter crosslinking time may be beneficial, enabling the 3-dimensional structure of interest to be printed rapidly with high shape fidelity.

Accordingly, in one aspect, there is provided a modified gelatin, wherein the gelatin is derived from a marine source and is modified to incorporate a crosslinkable group comprising a reactive moiety, the reactive moiety being reactive in a click chemical reaction.

In some embodiments, the number of proline residues in the gelatin is not more than 20% of the total number of amino acid residues in the modified gelatin.

In some embodiments, the number of hydroxyproline residues in the gelatin is not more than 20% of the total number of amino acid residues in the modified gelatin.

In some embodiments, the modified gelatin is characterizable by one or more of the following
a) wherein the reactive moiety content of the modified gelatin is from about 25 μmol/g to about 1,000 μmol/g;
b) wherein the reactive moiety content of the modified gelatin is from about 300 μmol/g to about 700 μmol/g;
c) wherein an aqueous solution of the modified gelatin of 20% wt/v or lower has a viscosity of below 100,000 mPa·s across a shear rate (l/s) of from 10 to 1,000; and
d) wherein an aqueous solution of the modified gelatin of 20% wt/v or lower has a complex viscosity of below 10⁸ mPa·s across a temperature range of from 0° C. to 40° C.

In some embodiments, the gelatin is a cold-water adapted fish gelatin. In some embodiments, the cold-water adapted fish is from a genus selected from the group consisting of Salmo, Gadus, Oncorhynchus and Merluccius, preferably Salmo or Oncorhynchus.

In some embodiments, the reactive moiety is selected from the group consisting of alkyne, amine, alkene, conjugated diene, thiol, isonitrile and tetrazine. In some embodiments, the reactive moiety is selected from the group consisting of amine, thiol, alkene, conjugated diene, azide and alkyne. In some embodiments, the reactive moiety is a thiol.

In some embodiments, the gelatin has been modified by a reaction selected from the group consisting of:
i) reaction of a gelatin amine group with Traut's reagent;
ii) reaction of a gelatin carboxylic acid group with a diamine;
iii) reaction of a gelatin carboxylic acid group with a diamine, and subsequent reaction with Traut's reagent;
iv) amide coupling of a gelatin carboxylic acid or carboxylate with an additional cysteine.

In another aspect, there is provided a process for producing a modified gelatin as defined herein, comprising reacting a gelatin derived from a marine source, with a crosslinkable group precursor.

In some embodiments, the gelatin derived from a marine source is reacted with a crosslinkable group precursor by:
i) reaction of a gelatin amine group with a crosslinkable group precursor which is Traut's reagent;
ii) reaction of a gelatin carboxylic acid group with a crosslinkable group precursor which is a diamine;
iii) reaction of a gelatin carboxylic acid group with a diamine, and subsequent reaction with Traut's reagent;
iv) amide coupling of a gelatin carboxylic acid or carboxylate with a crosslinkable group precursor which is cysteine.

In another aspect, there is provided a modified gelatin which is produced or producible by a process as defined herein.

In another aspect, there is provided a crosslinked modified gelatin, which is produced by reacting a modified gelatin as defined herein, in a click chemical reaction with a crosslinker comprising two or more further reactive moieties, the further reactive moieties being reactive in the click chemical reaction with the crosslinkable group reactive moiety incorporated into the modified gelatin.

In some embodiments, the crosslinker further reactive moieties are selected from the group consisting of alkene, alkyne and thiol. Examples of alkene further reactive moieties include maleimide, vinyl sulfone, acrylate, acrylamide and methacrylate. In some embodiments, the cross-linker further reactive moieties are selected from the group consisting of maleimide, vinyl sulfone, acrylate, acrylamide and methacrylate.

In some embodiments, the cross-linker has the formula:

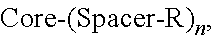

wherein Core is an atom or group providing an attachment for n spacer-R groups, Spacer is a spacer group, R is a group comprising a further reactive moiety, and n is an integer of from 2 to 8. In some embodiments, Core is C(—CH₂O—)₄; n is 4; Spacer is (—CH₂CH₂O—)ₘ, wherein m is an integer of from 2 to 150; and R is

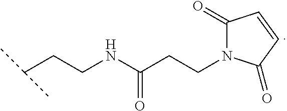

In some embodiments, Core is C(—CH₂O—)₄; n is 4; Spacer is (—CH₂CH₂O—)ₘ, wherein m is an integer of from 2 to 50; and R is

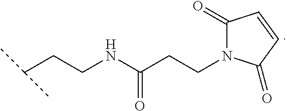

In some embodiments, Core is C(—CH₂O—)₄; n is 4; Spacer is (—CH₂CH₂O—)ₘ, wherein m is an integer of from 2 to 150; and R is

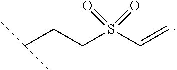

In another aspect, there is provided a process of producing a cross-linked modified gelatin as defined herein, comprising reacting a modified gelatin as defined herein, in a click chemical reaction with a crosslinker comprising two or more further reactive moieties, the reactive moieties being reactive in the click chemical reaction with the crosslinkable group reactive moiety incorporated into the modified gelatin.

In some embodiments, the process is carried out under ambient light conditions.

In another aspect, there is provided a crosslinked modified gelatin produced or producible by a method as defined herein.

In another aspect, there is provided a hydrogel comprising a crosslinked modified gelatin as defined herein, and water.

In some embodiments, the hydrogel is characterizable by one or more of the following:
a) wherein the hydrogel of 2.5% wt/v or greater has a relaxed mass swelling ratio of 15 or less;
b) wherein the hydrogel of between 2.5% and 10% wt/v has an equilibrium swelling ratio of from 10 to 25; and
c) wherein the hydrogel of between 2.5% and 10% wt/v has an equilibrium water content of from 80% to 100%.

In some embodiments, the hydrogel is characterizable by one or more of the following:
a) wherein the hydrogel of 2.5% wt/v or greater has a relaxed mass swelling ratio of 15 or less;
b wherein the hydrogel of between 2.5% and 10% wt/v has an equilibrium swelling ratio of from 10 to 25;
c) wherein the hydrogel of between 2.5% and 10% wt/v has an equilibrium water content of from 80% to 100%;
d) wherein during preparation the hydrogel of between 2.5% and 10% wt/v has a crosslinking time, at room temperature, of from 1 second to 10 seconds; and
e) wherein during preparation the hydrogel of between 2.5% and 10% wt/v has a crosslinking time, at room temperature, of from 10 minutes to 30 minutes.

In another aspect, there is provided a method of making a hydrogel, comprising admixing a crosslinked modified gelatin as defined herein, and water.

In another aspect, there is provided a method of making a hydrogel, comprising carrying out a method of making a crosslinked modified gelatin as defined herein, in the presence of water.

In another aspect, there is provided a hydrogel which is produced or producible by a process as defined herein.

In another aspect, there is provided use of a hydrogel as defined herein as a matrix for cell growth, or for 3D bioprinting.

In another aspect, there is provided a method of growing cells, comprising: providing a cell growth matrix comprising a hydrogel as defined herein; and growing cells in and/or on the cell growth matrix.

In another aspect, there is provided a kit for producing a cross-linked modified gelatin comprising:
a) a modified gelatin as defined herein; and
b) a crosslinker as defined herein.

### DESCRIPTION OF THE FIGURES

FIG. 1 shows example synthesis pathways for gelatin functionalization with click-reactive thiol groups: A—thiolation of native cold-water adapted fish gelatin using Traut's reagent to produce Traut's thiolated cold-water adapted gelatin; B—amination of native cold-water adapted fish gelatin using ethylenediamine and 1-Ethyl-3-(3-dimethylaminopropyl) carbodiimide (EDC) to produce aminated cold-water adapted fish gelatin; C—amination of native cold-water adapted fish gelatin using ethylenediamine and EDC to produce aminated cold-water adapted gelatin, followed by thiolation of aminated gelatin using Traut's reagent to produce two-step Traut's thiolated cold-water adapted fish gelatin.

FIG. 2 contains graphs representing the chemical and spectroscopic characteristics of native and click-reactive thiolated cold-water fish skin gelatin synthesized using various pathways: A—amine content of native cold-water adapted fish gelatin (in the Figure: “Native FG”), aminated cold-water adapted fish gelatin (in the Figure: “Aminated”), Traut's thiolated cold-water adapted gelatin (in the Figure: “Traut's”), two-step Traut's thiolated cold-water adapted fish gelatin (in the Figure: “Aminated Traut's”), and L-cysteine coupled cold-water adapted fish gelatin (coupling formed using EDC/NHS chemistry) (in the Figure: “EDC/NHS L-cys”); B—thiol content of Native FG, Traut's, Aminated Traut's and EDC/NHS L-cys cold-water adapted fish gelatins; C—¹H-NMR spectra of Native FG, Traut's, Aminated Traut's and EDC/NHS L-cys cold-water adapted fish gelatins.

FIG. 3 contains graphs representing the rheological properties of mammalian (porcine type A skin) gelatin (in the Figure: “Gelatin”) and thiolated cold-water adapted fish skin gelatin (in the Figure: “Gel-SH”) solutions of varying concentrations: A—viscosity over shear rate; B—complex viscosity over temperature for the gelatins from FIG. 3A (mean±SEM; n=3).

FIG. 4 contains graphs representing mechanical properties of hydrogels formed from thiolated cold-water adapted fish skin gelatin and PEG-4MAL: A—Stress-strain curves using three varying concentrations of thiolated cold-water adapted fish skin gelatin (in the Figure: “Gel-SH”), n=1; B—Young's modulus determined using hydrogel height, area, and slope of stress-strain curves at 10-15% compressive strain in (n=8; mean±SEM; ***=P≤0.0001, ****=P≤0.0001).

FIG. 5 shows the click hydrogel reaction scheme, graphs representing precursor pH and crosslinking time using various concentrations of HEPES (4-(2-hydroxyethyl)-1-piperazineethanesulfonic acid) buffer, and hydrogels: A—the hydrogel crosslinking schematic to produce Gel-SH/PEG-4MAL hydrogels from thiolated cold-water adapted fish skin gelatin (in the Figure: “Gel-SH”) and PEG-4MAL, crosslinked via Michael-type addition at a 1:1 thiol (in the Figure: “SH”) to maleimide (MAL) molar ratio; B—pH of hydrogel precursors and Gel-SH/PEG-4MAL hydrogels prepared using 100 mM, 200 mM and 300 mM HEPES buffer (mean±SEM; n=3); C—crosslinking times of Gel-SH/PEG-4MAL hydrogels with the Gel-SH concentration reported as final concentration within the hydrogel. (mean±SEM; n=8; significance testing=one-way ANOVA; ***=P≤0.0005); D—Gel-SH/PEG-4MAL hydrogels imaged via Nikon SMZ25 stereomicroscope with scale=1 cm.

FIG. 6 contains graphs representing swelling properties of Gel-SH/PEG-4MAL hydrogels: A—relaxed mass swelling ratio; B—equilibrium swelling ratio; C—equilibrium water content (n≤4; mean±SEM; significance testing=one-way ANOVA; **=P≤0.001, ***=P≤0.0001).

FIG. 7 contains images and graphs representing the viability of MCF-7 breast cancer cells in Gel-SH/PEG-4MAL click hydrogels as compared with photocrosslinkable gelatin methacryloyl hydrogels (in the Figure: “GelMA”): A—maximum intensity projections of live (FDA) and dead (PI) MCF-7 breast cancer cells encapsulated in Gel-SH/PEG-4MAL hydrogels at encapsulation density 1×10⁶ cells/mL, with scale=500 μm; B—viability of MCF-7 cell-laden Gel-SH/PEG-4MAL hydrogels (gel-SH concentration (% wt/v) reported as final concentration of thiolated cold-water adapted fish skin gelatin in Gel-SH/PEG-4MAL hydrogels. N=3. Significance testing=One-way ANOVA. *=P≤0.05. Error bars=SEM).

FIG. 8 contains images representing spheroid formation of MCF-7 breast cancer cells encapsulated in Gel-SH/PEG-4MAL hydrogels: Encapsulated MCF-7 nuclei (DAPI) and F-actin (Phalloidin) visualized via confocal microscopy and maximum intensity projections (Gel-SH concentration reported as final Gel-SH % (wt/v) in click hydrogel. Imaging via Leica SP5 confocal microscope. N=1. Objective=4×. Scale bar=250 μm).

FIG. 9 shows that crosslinking time and gel formation can be controlled by selection of the click-reactive PEG moiety. Here, Gel-SH hydrogels crosslinked with 4-arm PEG-vinyl sulfone (PEG4-4VS) crosslinked slower than gels crosslinked with PEG-4MAL, offering extended liquid handling time.

FIG. 10 shows the evaluation of MCF-7 breast cancer cell viability and metabolic activity in Gel-SH hydrogels crosslinked with PEG-4MAL or PEG-4VS. A—Brightfield microscopy images illustrating the growth and morphology of MCF-7 cells encapsulated in 10% (w/v) Gel-SH hydrogels with PEG-4MAL or PEG-4VS at day 1 and day 7 of culture. B—Quantitative assessment of MCF-7 cell metabolic activity encapsulated in 10% (w/v) Gel-SH hydrogels using PrestoBlue™ Cell Viability Reagent (ThermoFisher Scientific) at day 1 and day 7. C—Metabolic activity of MCF-7 cells encapsulated in hydrogels containing either 5% or 10% (w/v) Gel-SH and equimolar concentrations of PEG-4VS, assessed at day 1 and day 7 of culture.

### DETAILED DESCRIPTION

### Definitions

Unless defined otherwise, all technical and scientific terms used herein have the same meaning as commonly understood by those of ordinary skill in the field of the present disclosure. Although any methods and materials similar or equivalent to those described herein can be used in the practice or testing of the present disclosure, preferred methods and materials are described. For the purposes of the present disclosure, a number of terms are defined herein.

The present disclosure refers to the entire contents of certain documents being incorporated herein by reference.

It is to be understood that if any prior art publication is referred to herein, such reference does not constitute an admission that the publication forms a part of the common general knowledge in the art.

As used herein, the term “and/or”, e.g., “X and/or Y” shall be understood to mean either “X and Y” or “X or Y” and shall be taken to provide explicit support for both meanings or for either meaning.

The term “about” as used herein, when referring to a numerical value or range, allows for a degree of variability in the value or range, for example within 10% of a stated limit of a range.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Unless otherwise indicated, terms such as “first,” “second,” etc. are used herein merely as labels, and are not intended to impose ordinal, positional, or hierarchical requirements on the items to which these terms refer. Moreover, reference to a “second” item does not require or preclude the existence of lower-numbered item (e.g., a “first” item) and/or a higher numbered item (e.g., a “third” item).

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example and without limitation, two of item A, one of item B, and ten of item C; four of item B and seven of item C; or some other suitable combination.

As used herein, the word “comprise” and other forms of the word, such as “comprising” and “comprises,” means including but not limited to, and is not intended to exclude, for example, other additives, components, integers, or steps.

A “protein” as used herein is a polymer of amino acid residues and includes a peptide including an oligopeptide.

A “gelatin” as used herein is a protein obtainable by the at least partial hydrolysis of collagen.

“Collagen” as used herein is the main structural protein of connective tissues in animals, found most often in animal cartilage, bones, tendons, ligaments, and skin.

“Marine source” as used herein refers to a marine species animal.

A “cold-water adapted marine species” as used herein is a marine species animal with a natural habitat in waters with an average temperature of 22° C. or below and is as distinct from a land animal.

A “fish” as used herein is a marine species animal with a backbone, gills and fins, and includes mammals fitting this description such as whales, but is as distinct from a land mammal.

A “click chemical reaction” as used herein refers to the concept introduced by K. Barry Sharpless of The Scripps Research Institute which describes chemical reactions which proceed under a high thermodynamic driving force between reactive functional groups to form covalently bonded connector functional groups. They are generally high yielding, are often stereospecific, may generate non-toxic byproducts, and may proceed in the presence of water. See for example Nair et al. For the avoidance of doubt, a reaction involving the photoinitiated crosslinking of methacryloyl groups is not a click chemical reaction. In some embodiments, the click chemical reaction is a reaction which is not photoinitiated. In some embodiments, the click chemical reaction is a reaction that can be carried out under ambient light conditions. In some embodiments, the click chemical reaction is a reaction that can be carried out in the dark.

A “classical click chemical reaction” as used herein is a click chemical reaction in the from of a [3+2] cycloaddition, a [4+1] cycloaddition, a thiol-ene reaction, a thiol-yne reaction, a Diels-Alder cycloaddition (between a conjugated diene and an alkene), nucleophilic substitution or addition reactions across alkenes or alkynes, and the formation of a thiol ether or disulfide bond. Additionally, this category encompasses Michael addition reactions, where a nucleophile such as a thiol adds to the double bond of an α,β-unsaturated carbonyl compound.

### Modified Gelatins

Processes for producing native gelatin by the at least partial hydrolysis of collagen are generally known in the art. They are generally chemical or enzymatic processes. Chemical processes often use mild acid or alkaline treatments. Typical processes result in partially hydrolyzed collagen (gelatin) molecules of molecular weight from 5 kDa to 200 kDa.

The present disclosure is directed towards inter alia marine-source gelatins which have been modified to incorporate a crosslinkable group comprising a reactive moiety which is reactive in a click chemical reaction with a crosslinker containing a suitable further reactive moiety, and hydrogels formed by their crosslinking.

The gelatin is modified to incorporate a crosslinkable group comprising a reactive moiety. In other words, the gelatin has been reacted with an additional chemical species not originally part of the gelatin.

The crosslinkable group which is incorporated into the gelatin to provide a modified gelatin may be incorporated by reaction of a chemical species consisting of or comprising the crosslinkable group with the gelatin molecule. In many embodiments, the gelatin is modified such that the pendent group is covalently bound to the gelatin.

That is, a gelatin molecule and crosslinkable group chemical species are chemical species which, in the case of forming covalent bonds, contain functional groups consisting of or comprising reactive moieties, which react together to form covalent bonds in connecting functional groups. Specifically, a reaction between a gelatin molecule and crosslinkable group chemical species may occur between an exposed reactive moiety of the gelatin molecule and a complementary reactive moiety of the crosslinkable group chemical species to form a modified gelatin.

Reactive moieties which participate in chemical reactions are known or determinable by those skilled in the art and many are described below. The reactions described herein may be assisted by a facilitator such an added energy source (e.g. light, heat) or additional reagent (e.g. catalyst, base, acid, initiator, coupling agent or other). Assisted chemical reactions are known or determinable by those skilled in the art. An example is the copper-catalyzed azide-alkyne click chemistry cycloaddition reaction between azide and alkyne moieties. Another is amide bond formation between carboxylic acids and amines using coupling agents. Various coupling agents are known in the art. Reaction conditions including temperature, pressure and other physical parameters, the use of e.g. solvents, purification and characterization methods, are known or determinable by those skilled in the art.

Gelatin from natural sources of cold-water adapted marine species such as, but not limited to, the Salmo or Oncorhynchus genus differ from land mammal gelatins in their amino acid composition, in that they usually contain 20% or less proline, and 20% or less hydroxyproline with respect to total amino acid content, whereas land mammal gelatins contain around 30% of each of these amino acids. These native gelatins also differ from their counterparts derived from warm-water adapted marine species, which generally contain around 25% of each of these amino acids. The relatively decreased proline and hydroxyproline content of these native gelatins as compared to land mammal gelatin and warm-water marine species gelatins and derivatives results in a significantly lower melting point (approximately 4° C.), as well as significantly lower, stable rheological properties over a wide temperature window between approximately 15-40° C. This gives to rise to various properties which may be used to advantage in various applications, including, as the present inventors have found, in producing hydrogels from modified cold-water adapted marine species gelatins containing click chemical reactive moieties.

In preferred embodiments, the number of proline and/or hydroxyproline residues is not more than 20% of the total number of amino acid residues in the modified gelatin. Preferably, the proline and/or hydroxyproline content is less than 20%, being 19% or less, 18% or less, 17% or less or 16% or less, of the total number of amino acid residues in the modified gelatin.

In some embodiments, the gelatin comprises Ala, Gly, Pro and 4-Hyp. In some embodiments, at least half of the amino acids present in the gelatin are selected from the group consisting of Ala, Gly, Pro and 4-Hyp.

In some embodiments, the gelatin contains: from 8-14 mol % Ala; from 30-40 mol % Gly; from 4-10 mol % 4-Hyp; and from 10-16 mol % Pro.

In some embodiments, the gelatin contains: from 8-14 mol % Ala; from 30-40 mol % Gly; from 4-10 mol % 4-Hyp; from 10-16 mol % Pro; and the remaining amino acids being selected from the group consisting of Arg, Asp, Glu, His, Ile, Leu, Lys, Met, Phe, Ser, Thr and Val.

In some embodiments, the gelatin comprises Ala, Gly, Pro, 4-Hyp and Glu. In some embodiments, at least half of the amino acids present in the gelatin are selected from the group consisting of Ala, Gly, Pro, 4-Hyp and Glu.

In some embodiments, the gelatin contains: from 8-13 mol % Ala; from 7-11 mol % Glu; from 21-37 mol % Gly; from 5-10 mol % 4-Hyp; and from 8-12 mol % Pro.

In some embodiments, the gelatin contains: from 9-12 mol % Pro; from 7-9 mol % 4-Hyp; from 10-12 mol % Glu; from 21-23 mol % Gly; and from 9-10 mol % Ala.

In some embodiments, the gelatin contains: from 9-12 mol % Pro; from 7-9 mol % 4-Hyp; from 10-12 mol % Glu; from 21-23 mol % Gly; from 8-9 mol % Arg; from 9-10 mol % Ala; and not more than 7 mol % of any other amino acid.

The “cold-water adapted marine species” from which the native gelatin may be derived may be a species of the genus Salmo including Salmo salar, the genus Oncorhynchus including Oncorhynchus gorbuscha, Oncorhynchus tshawytscha, Oncorhynchus keta, Oncorhynchus kisutch, Oncorhynchus masou and Oncorhynchus nerka, the genus Gadus including Gadus chalcogrammus, Gadus morhua and Gadus microcephalus, the genus Melanogrammus including Melanogrammus aeglefinus or the genus Merluccius. In preferred embodiments, the native gelatin is derived may be a species of the genus Salmo and in particular the species Salmo salar.

The intended purpose of the reactive moiety which is reactive in a click chemical reaction is to undergo a click chemical reaction with a crosslinkable group precursor to form crosslinks via connector functional groups between gelatin molecules to form a hydrogel. The reactive moiety may be selected based on a desired click chemical reaction, or the click chemical reaction may be dictated by the selection of a particular reactive moiety.

Click chemical reactions and their reactive functional groups are known in the art. Examples include [3+2] cycloadditions such as the Huisgen 1,3-dipolar cycloaddition or azide-alkyne cycloaddition between azide and alkyne moieties, amide bond coupling between amines and carbonyl groups (e.g. ester, carboxylic acid, aldehyde, anhydride, ketone, acyl halide) including via reductive amination, Diels-Alder cycloadditions between alkenes and conjugated dienes, ester bond formation between alcohols or alkoxides and carbonyl groups, ester bond formation between alcohols and nitriles, thioether bond formation between thiols and alkyl halides or alkenes, disulfide bond coupling between thiols, alkenyl sulfide bond formation between thiols and alkynes, other nucleophilic substitution reactions especially between epoxides and alcohols, amines or organometallic nucleophiles such as organolithium compounds, Michael addition between thiols and alkenes, other nucleophilic addition reactions especially between carbonyl groups and alcohols, alkoxides, amines and organometallic nucleophiles, oxime or nitrone formation between alkoxyamines and aldehydes or ketones, [4+1] cycloadditions between isonitrile and tetrazines.

Accordingly, representative reactive moieties of the crosslinkable group of the modified gelatin include alkynes, amines, carbonyl groups, alkenes including conjugated dienes, alcohols, alkoxides, nitriles, thiols, alkyl halides, epoxides, organometallic species, alkoxyamines, isonitrile and tetrazines.

In preferred embodiments, the crosslinkable group is intended to undergo a classical click chemical reaction. In these embodiments, the reactive moiety is preferably selected from the group consisting of an alkyne, amine, alkene, conjugated diene, thiol, isonitrile and tetrazine. In more preferred embodiments, the crosslinkable group is intended to undergo a Michael addition reaction between a thiol and an alkene or a thiol and an alkyne, disulfide bond coupling between thiols, a Diels-Alder cycloaddition between an alkene and a conjugated diene, or an azide-alkyne cycloaddition between an azide and an alkyne, in which case the reactive moiety is preferably selected from the group consisting of an alkyne, alkenes, conjugated diene and a thiol. In most preferred embodiments, the crosslinkable group is intended to undergo a Michael addition reaction between a thiol and an alkene, in which case the reactive moiety is preferably selected from the group consisting of an alkene and a thiol.

As used herein, the mention of a particular reactive moiety is taken to encompass chemical functional groups which contain that moiety, whether the functional group comprises or consists entirely of the reactive moiety. For instance, an amine reactive moiety may refer to the functional groups or primary, secondary or tertiary amine, amide, guanidine, hydrazine, hydrazine and other functional groups which comprise an amine portion.

The crosslinkable group which is incorporated into the gelatin to provide a modified gelatin may be incorporated by the reaction of a crosslinkable group chemical species with an exposed reactive moiety of the gelatin molecule, to form a covalent bond.

Generally speaking, gelatin comprises the amino acids proline and/or hydroxyproline and others which may be of the 20 known common amino acids, and in particular glycine, glutamic acid, arginine, alanine, aspartic acid and cysteine. The amino acids of gelatin may contain functional groups having reactive moieties which are exposed (i.e. available) for reaction with a complementary reactive moiety of a functional group of the crosslinkable group species. The exposed reactive moieties may be, for example hydroxyl groups (hydroxyproline, serine, threonine), amines or amides (arginine, lysine, asparagine, glutamine, and terminal amino acids from the hydrolysis of collagen), thiols (cysteine) and carboxylic acids (glutamic acid, aspartic acid, and terminal amino acids from the hydrolysis of collagen).

Accordingly, in preferred embodiments, in addition to providing a reactive moiety in a click chemical reaction, the crosslinkable group chemical species contains a second reactive moiety which is reactive with an exposed reactive moiety of a gelatin molecule. As the available exposed reactive moieties of gelatin molecules are commonly hydroxyl groups, amines or amides, thiols and carboxylic acids, preferably the complementary reactive moiety of the crosslinkable group species is reactive with one or more of these functional groups. In these embodiments, the complementary reactive moiety is preferably carboxylic acid (for reaction with a hydroxyl, amine), epoxide (for reaction with an hydroxyl, amine, amide), anhydride (for reaction with an hydroxyl, amine, amide), aldehyde (for reaction with an hydroxyl, amine, amide), ketone (for reaction with an hydroxyl, amine, amide), ester (for reaction with an hydroxyl, amine, amide), isocyanate (for reaction with an hydroxyl, amine), isothiocyanate (for reaction with an hydroxyl, amine), thioimidate (for reaction with an amine) or acyl halide (for reaction with an hydroxyl, amine), alkyne (for reaction with an amine, thiol), alkene (for reaction with a thiol), or amine, amide, hydroxyl, epoxide, anhydride, aldehyde, ketone, ester, isocyanate, isothiocyanate, acyl halide, alkyne or alkene, for reaction with exposed reactive moieties as indicated in the foregoing.

The click chemical groups as described herein may also be equally applicable to the exposed reactive moiety of the gelatin molecule and the complementary reactive moiety of the crosslinkable group species.

When crosslinking of gelatin molecules by the crosslinkable group species itself may occur, this may be avoided in a number of ways, typically by selection of appropriate crosslinkable group species or reaction conditions or agents, as appropriate. In representative examples, the crosslinkable group species may comprise only one complementary reactive moiety, or it may contain two or more wherein all but one contain a protecting group which may be removed after the exposed reactive moiety of the gelatin molecule and the complementary reactive moiety of the crosslinkable group species have reacted.

In preferred embodiments, the exposed reactive moiety is an amine, carbonyl group or thiol. Preferably, the complementary reactive moiety is (for reaction with an amine) a carbonyl group, epoxide, thioimidate, isocyanate or isothiocyanate, (for reacting with a carboxylic acid) a hydroxyl or amine, or (for reacting with a thiol) a thiol, alkene or alkyne. Preferably, the exposed reactive moiety is an amine (preferably of guanidine) and the complementary reactive moiety is a thioimidate, and/or the exposed reactive moiety is a carbonyl group (preferably carboxylic acid) and the complementary reactive moiety is an amine (preferably a primary amine).

The crosslinkable group species may thus essentially be a di-functionalized molecule, meaning that it contains a reactive moiety for modifying gelatin, and a reactive moiety for click chemical crosslinking with a crosslinker. The crosslinkable group species is not otherwise particularly limited in structure. The structure of the di-functionalized molecule may otherwise consist of or comprise, for example, an optionally further functionalized (e.g. containing a further functional group such as a functional group as herein described) alkylene, alkenylene or alkynylene, which may preferably be a C₁-C₁₀ alkylene, C₂-C₁₀ alkenylene, C₂-C₁₀ alkynylene, preferably a C₁-C₆ alkylene, C₂-C₆ alkenylene, C₂-C₆ alkynylene. In preferred embodiments, the structure of the di-functionalized crosslinkable group species otherwise consists of an unfunctionalized C₁-C₁₀ alkylene, preferably an unfunctionalized C₁-C₆ alkylene.

Example preferred crosslinkable group species are Traut's reagent (2-Iminothiolane), other cyclic thioimidates, ethylene diamine, propylene diamine etc., ethylene glycol, propylene glycol etc., malonic acid, succinic acid etc., amino acids such as cysteine, lactones, and lactams.

An intermediate step that may be performed in forming a modified gelatin involves the treatment of a gelatin with a first crosslinkable group species comprising a reactive moiety which is reactive with a complementary reactive moiety of a second crosslinkable group species, the second crosslinkable group species comprising a reactive moiety which is reactive in a click chemical reaction. For instance, a gelatin molecule may be reacted with a first crosslinkable group species, the reaction taking place between one type of exposed reactive moiety of the gelatin, using a species that provides an exposed crosslinkable group reactive moiety that is the same as another type of exposed reactive moiety of the gelatin, followed by reaction of the exposed reactive moieties with a second crosslinkable group species that comprising a reactive moiety for a click chemical reaction with a crosslinker. This has the advantage of allowing the content of the reactive moiety for click chemical crosslinking to be increased and allows control of the extent of crosslinking formed in preparing a hydrogel.

A crosslinkable group species may be referred to as a “crosslinkable group precursor”.

In one example, exposed carboxylic acid groups of a gelatin, but not the amines, are reacted with a first crosslinkable group species which provides an exposed amine reactive moiety, and the amines of the native gelatin and first crosslinkable group are reacted with a second crosslinkable group species which provides an exposed reactive moiety for click chemical reaction with a crosslinker. This represents a preferred embodiment comprising the following steps: i) reaction of a (native) gelatin exposed carboxylic acid group with a diamine; and ii) subsequent reaction (of the native exposed amines and diamine-exposed amines) with Traut's reagent.

The applicable reactive moieties may be as herein described. In a specific example, the reactive moiety content may be increased relative to that of the native gelatin by the treatment of a gelatin with ethylenediamine (the first crosslinkable group species) which reacts with carboxylic acids and increases the exposed amine content, followed by treatment with Traut's reagent which reacts with amines to provide a high content of thiol moieties for click chemical reaction with a crosslinker.

Third and subsequent treatments with crosslinkable group species are similarly applicable.

Another intermediate step that may be performed in forming a modified gelatin involves the chemical conversion of an exposed reactive moiety from one type into another. Another intermediate step that may be performed in forming a modified gelatin involves the chemical conversion of a reactive moiety of the crosslinkable group from one type into another. For example, the reactive moiety may be converted into another as may be applicable to a particular click chemical reaction with a crosslinker. Another intermediate step that may be performed is the addition or removal of protecting groups, as appropriate. Intermediate steps assist to provide flexibility in the reagents selected for the crosslinkable group species and the crosslinker. Intermediate steps may involve treating a gelatin or a modified gelatin with a reactive reagent. Examples of chemical conversion of functional groups with reactive reagents include the conversion of an epoxide to an amine using ammonia reagent, and conversion of an epoxide to a hydroxide using sulphuric acid reagent. Many other examples are known or determinable to the person skilled in the art.

In some embodiments, the modified gelatin is a gelatin that has been modified by reaction of carboxylic acid groups present in unmodified gelatin with a crosslinkable group precursor comprising an amine group (which may for example react with a carboxylic acid group present in the gelatin). For example, the gelatin may have been modified by reaction with an alkylene diamine (e.g. a C₂₋₆alkylenediamine such as ethylenediamine or propylene diamine), or a monoprotected alkylene diamine which is subsequently deprotected, to introduce a crosslinkable group via amide bond formation. In such embodiments, the crosslinkable group may have the formula —NH—C₂₋₆alkylene-NH₂. As another example, the gelatin may have been modified by reaction with a group of the formula H₂N—C₂₋₆alkylene-S-PG, wherein PG represents a protecting group for a thiol, such that following deprotection the crosslinkable group may have the formula —NH—C₂₋₆alkylene-SH.

In some embodiments, the gelatin may have been modified by reaction with a crosslinkable group precursor comprising a carboxylic acid group (which may for example react with an amino group present in the gelatin). For example, the gelatin may have been modified by reaction with a group of the formula HO₂C—C₂₋₆alkylene-NH-PG, where PG represents a protecting group for an amine, followed by deprotection of the protecting group, to introduce a crosslinkable group via amide bond formation. In another example, the gelatin may have been modified by reaction with a group of the formula HO₂C—C₂₋₆alkylene-S-PG, where PG represents a protecting group for a thiol, followed by deprotection of the protecting group, again to introduce a crosslinkable group via amide bond formation.

In some embodiments, the gelatin may have been modified by reaction with a crosslinkable group which is an amino acid or protected amino acid, which contains a first functional group which is capable of reacting with a reactive group present in the gelatin, and which contains a second functional group, or a protected form of a second functional group, which second functional group constitutes a reactive moiety which is reactive with a reactive moiety in the crosslinker. An example of such an amino acid is cysteine, e.g. L-cysteine.

The modified gelatin may be characterizable by one or more properties, which may be a reactive moiety content and a viscosity as described in the following.

The modified gelatin may be characterizable by a reactive moiety content of from about 25 μmol/g to about 1,000 μmol/g, preferably about 50 or 100 μmol/g to about 950 μmol/g, preferably about 150 or 200 μmol/g to about 900 μmol/g, preferably about 250 or 300 μmol/g to about 850 μmol/g and preferably about 350 or 400 μmol/g to about 850 μmol/g. This is preferred when the reactive moiety is an amine or a thiol.

The modified gelatin may be characterizable by a reactive moiety content of from about 300 μmol/g to about 700 μmol/g, preferably about 350 μmol/g to about 650 μmol/g, preferably about 400 μmol/g to about 600 μmol/g, and preferably about 450 μmol/g to about 550 μmol/g. This is preferred when the reactive moiety is a thiol.

The Examples below describe a method for measuring reactive moiety content, which represents the preferred method.

The modified gelatin may be characterizable by a viscosity, wherein an aqueous solution of the modified gelatin of 20% wt/v or lower, preferably of about 5% wt/v to about 20% wt/v, has a viscosity of below 100,000 mPa·s across a shear rate (l/s) of from 10 to 1,000, preferably a viscosity of below 10,000 mPa·s across a shear rate (l/s) of from 10 to 1,000, preferably a viscosity of below 1,000 mPa·s across a shear rate (l/s) of from 10 to 1,000, preferably a viscosity of below 100 mPa·s across a shear rate (l/s) of from 10 to 1,000, and preferably a viscosity of below 50 mPa·s across a shear rate (l/s) of from 10 to 1,000. The modified gelatin may also be characterizable by a viscosity, wherein an aqueous solution of the modified gelatin of 10% wt/v or lower, preferably of about 5% wt/v to about 10% wt/v, has a viscosity of below 10 mPa·s across a shear rate (l/s) of from 10 to 1,000. The modified gelatin may also be characterizable by a viscosity, wherein an aqueous solution of the modified gelatin of 5% wt/v or lower, preferably of about 5% wt/v, has a viscosity of below 5 mPa·s across a shear rate (l/s) of from 10 to 1,000. These viscosities, including in combination, are preferred when the crosslinkable group is formed using Traut's reagent and the reactive moiety is a thiol, and especially when the native gelatin is first functionalized with ethylenediamine (i.e. where ethylenediamine is used in an intermediate step as a first crosslinkable group species).

The Examples below describe a method for measuring viscosity, which represents the preferred method.

The modified gelatin may be characterizable by a complex viscosity, wherein an aqueous solution of the modified gelatin of 20% wt/v or lower, preferably of about 5% wt/v to about 20% wt/v, has a complex viscosity of below 10⁸ mPa·s across a temperature range of from 0° C. to 40° C., preferably a complex viscosity of below 10⁷ mPa·s across a temperature range of from 0° C. to 40° C., and preferably a complex viscosity of below 10⁶ mPa·s across a temperature range of from 20° C. to 40° C. The modified gelatin may also be characterizable by a complex viscosity, wherein an aqueous solution the modified gelatin of 10% wt/v or lower, preferably of about 5% wt/v to about 10% wt/v, has a complex viscosity of below 10⁶ mPa·s across a temperature range of from 0° C. to 40° C. These complex viscosities, including in combination, are preferred when the crosslinkable group is formed using Traut's reagent and the reactive moiety is a thiol, and especially when the native gelatin is first functionalized with ethylenediamine.

The Examples below describe a method for measuring complex viscosity, which represents the preferred method.

Gelatins and modified gelatins as disclosed herein may be present in the form of salts. In many cases, groups present in the gelatins are capable of forming acid and/or base salts by virtue of the presence of amino and/or carboxyl groups or groups similar thereto. Salts can be formed with inorganic acids and organic acids and bases. Examples include salts with hydrochloric acid, sulfuric acid and the like, acetic acid, propionic acid and the like, sodium, potassium bases and the like, and amines. Many others are known in the art.

### Crosslinked Gelatins and Hydrogels

A crosslinked modified gelatin may be produced by reacting a modified gelatin as described herein, in a click chemical reaction with a crosslinker, where the crosslinker comprises two or more reactive moieties, the reactive moieties being reactive in the click chemical reaction with the reactive chemical moieties of the crosslinkable group of the modified gelatin.

A benefit of the crosslinked modified gelatins and hydrogels as described herein arises from ease and speed of formation by click chemical reaction, and the biocompatible nature of many click chemical reactions, which generally do not require any or significant external inputs to initiate. For instance, as compared to photoinitiated crosslinking of gelatins modified to contain methacryloyl groups, production of the crosslinked modified gelatins and hydrogels as described herein may proceed in the absence of photoinitiation and in the absence of agents such as free-radical scavengers which are often required in photoinitiated chemical reactions, and without the need for specialty equipment.

Accordingly, in preferred embodiments, the reaction is carried out under ambient light conditions.

An additional benefit is the ability to tune the crosslinking time for the formation of a hydrogel, by use of different crosslinkers. For example, a crosslinker may contain maleimide groups as the two or more reactive moieties, which will be reactive in a click chemical reaction with a reactive chemical moiety on the modified gelatin, for example such as a thiol. Such systems may have a relatively short crosslinking time (for example, less than 1 minute, or less than 30 seconds, or less than about 10 seconds), at room temperature. In contrast, a crosslinker which contains, for example, vinyl sulfone groups as the two or more reactive moieties may in some embodiments provide for a longer crosslinking time, (for example 10 minutes or more, or 20 minutes or more, or about 30 minutes). An advantage of having a short crosslinking time is if setting and hydrogel formation is desired quickly, for example in 3D printing applications. On the other hand, an advantage associated with longer crosslinking time (for example, more than about 10 minutes) is that this may be suitable for applications where more time to handle and process the components is required, for example during manual or automated liquid handling.

In some embodiments the modified gelatin and crosslinker are selected to provide a crosslinking time in the range of from 1 second to 1 minute, or from 1 second to 30 seconds, or from 1 second to 10 seconds. In some embodiments, the modified gelatin and crosslinker are selected to provide a crosslinking time in the range of from 1 minute to 5 minutes. In some embodiments, the modified gelatin and crosslinker are selected to provide a crosslinking time in the range of from 5 minutes to 1 hour, or from 10 minutes to 1 hour, or from 20 minutes to 1 hour, or from 30 minutes to 1 hour, or from 10 minutes to 30 minutes, or from 10 to 20 minutes, or from 20 minutes to 20 minutes.

The click chemical reaction between the crosslinkable group reactive moiety and the crosslinker reactive moiety forms covalent bonds. That is, a modified gelatin molecule comprising a crosslinker are chemical species which contain functional groups consisting of or comprising reactive moieties, which react together in click chemical reactions to form covalent bonds in connecting functional groups. Specifically, the click chemical reaction between the crosslinkable group of the modified gelatin and the crosslinker may occur between the reactive moiety of the crosslinkable group and a reactive moiety of the crosslinker.

As described above, the reactive moieties which participate in the click chemical reaction may be selected based on a desired click chemical reaction, or the click chemical reaction may be dictated by the selection of a particular reactive moiety. Click chemical reactions and their reactive functional groups are known in the art. Many examples are described above, including applicable representative example.

In preferred embodiments, the crosslinkable group and crosslinker are intended to undergo a classical click chemical reaction. In these embodiments, the reactive moiety of the crosslinkable group is selected from the group consisting of an alkyne, amine, alkene, conjugated diene, thiol, isonitrile and tetrazine. In which case, the preferred reactive moiety of the crosslinker is selected from the group consisting of an azide or thiol (for reacting with an alkyne), a carbonyl group or epoxide (for reacting with an amine), a conjugated alkene or thiol (for reacting with alkenes, including for example α,β-unsaturated carbonyl systems including maleimide, and vinyl sulfones), an alkene (for reacting with a conjugated diene or a thiol), a thiol (for reacting with thiols, alkyl halides, or alkenes, including for example α,β-unsaturated carbonyl systems including maleimide, and vinyl sulfones), isonitrile (for reacting with tetrazine) and tetrazine (for reacting with isonitrile).

In more preferred embodiments, the crosslinkable group and crosslinker are intended to undergo a Michael addition reaction between a thiol and an alkene or a thiol and an alkyne, disulfide bond coupling between thiols, a Diels-Alder cycloaddition between an alkene and a conjugated diene, or an azide-alkyne cycloaddition between an azide and an alkyne. Where in preferred embodiments the reactive moiety of the crosslinkable group is an alkyne, alkenes, conjugated diene or a thiol, in preferred embodiments the reactive moiety of the crosslinker is an azide, thiol, alkene or an alkene, respectably. Where in most preferred embodiments the reactive moiety of the crosslinkable group is intended to undergo a Michael addition reaction between a thiol and an alkene, the reactive moiety of the crosslinkable group is a thiol and the reactive moiety of the crosslinker is an alkene. In preferred embodiments, the functional group comprising the alkene reactive moiety is selected from the group consisting of maleimide, vinyl sulfone, acrylate, acrylamide and methacrylate.

As the crosslinker is intended to form at least one bond with at least two modified gelatin molecules, the crosslinker comprises two or more reactive moieties which are reactive in the click chemical reaction with the reactive chemical moieties of the crosslinkable group of the modified gelatin.

In some embodiments, the click chemical reaction is between a thiol reactive group present on the crosslinkable group, and an alkene reactive moiety present on the crosslinker.

In some embodiments, the click chemical reaction is between a thiol reactive group present on the crosslinkable groups, and either a vinyl sulfone or a maleimide reactive group present on the crosslinker.

The crosslinker species is thus essentially at least a difunctionalized molecule, and may be a tri-, tetra-etc. functionalized molecule, meaning that it contains at least two reactive moieties for click chemical crosslinking with the modified gelatin. The crosslinker species is not otherwise particularly limited in structure. It may otherwise consist of or comprise, for example, an optionally further functionalized (e.g. containing a further functional group such as a functional group as herein described) alkylene, alkenylene, alkynylene, alkyloxylene, alkenyloxylene, alkynyloxylene, or combinations thereof. The structure between the reactive moieties or functional groups comprising them, may be referred to as a “spacer”.

In preferred embodiments, the crosslinker has the following formula:

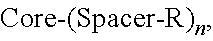

wherein Core is an atom or group providing an attachment for n spacer-R groups, Spacer is a spacer group, R is a functional group comprising a reactive moiety, and n is an integer.

In preferred embodiments, n is an integer of from 2 to 8, for example n may be 2, 3, 4, 5, 6, 7 or 8. Preferably, the crosslinker is tetra-functionalized, in that it contains four reactive moieties for click chemical crosslinking with the modified gelatin. In which case, in preferred embodiments, n is 4.

It may otherwise consist of or comprise, for example, an optionally further functionalized (e.g. containing a further functional group such as a functional group as herein described) alkylene, alkenylene, alkynylene, alkyloxylene, alkenyloxylene, alkynyloxylene, or combinations thereof.

In preferred embodiments, Core consists of a central tetra-substituted carbon atom having the formula C(Link)₄, wherein Link is an alkylene, alkenylene, alkynylene, alkyloxylene, alkenyloxylene or alkynyloxylene, which may preferably be a C₁-C₆ alkylene, C₂-C₆ alkenylene, C₂-C₆ alkynylene, C₁-C₆ alkyloxylene, C₂-C₆ alkenyloxylene or C₂-C₆ alkynyloxylene, preferably a C₁-C₆ alkyloxylene. In preferred embodiments. Link is —CH₂O—, in which case, in preferred embodiments, Core is C(—CH₂O—)₄ and n is 4.

In preferred embodiments, Spacer consists of an alkylene, alkenylene, alkynylene, alkyloxylene, alkenyloxylene or alkynyloxylene, preferably an alkyloxylene. In preferred embodiments, Spacer is (—CH₂CH₂O—)ₘ, wherein m is an integer. In these embodiments, Spacer may be referred to as polyethylene glycol (PEG). In some preferred embodiments, m is an integer of from 2 to 150, preferably from about 25 to about 140, preferably from about 50 to about 130, preferably from about 75 to about 120, or preferably about 100 to about 115. In some other preferred embodiments, m is an integer of from 2 to 50.

R is a group comprising the further reactive moiety. For example, R may contain a connecting portion, providing connectivity between the further reactive moiety and the Spacer, as well as the further reactive moiety itself. R may for example be an optionally further functionalized (e.g. containing a further functional group such as a functional group as herein described, preferably an amide bond) alkylene, alkenylene, alkynylene, alkyloxylene, alkenyloxylene or alkynyloxylene, further comprising the reactive moiety, which may preferably be a C₁-C₁₀ alkylene, C₂-C₁₀ alkenylene, C₂-C₁₀ alkynylene, C₁-C₁₀ alkyloxylene, C₂-C₁₀ alkenyloxylene or C₂-C₁₀ alkynyloxylene, further comprising the reactive moiety, preferably a C₁-C₁₀ alkylene further comprising the reactive moiety.

In preferred embodiments, R is

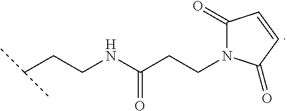

In other preferred embodiments, R is

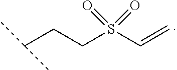

A representative preferred embodiment of the crosslinker is the species known as

PEG-4 maleimide (PEG4-MAL). PEG4-MAL has the structure Core-(Spacer-R)ₙ, wherein Core is C(—CH₂O—)₄, Spacer is (—CH₂CH₂O—)ₘ, wherein m is an integer of from 2 to 150, or from 25 to 150, or from 75 to 125, and R is

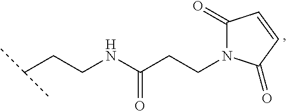

and is available from, for example, JenKem®.

Another representative preferred embodiment of the crosslinker is the species known as PEG-4 vinyl sulfone (PEG-4VS). PEG-4VS has the structure Core-(Spacer-R) n, wherein Core is C(—CH₂O—)₄, Spacer is (—CH₂CH₂O—)ₘ, wherein m is an integer of from 2 to 150, or from 25 to 150, or from 75 to 125, and R is

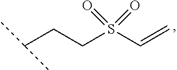

and is available from, for example, JenKem®.

The alkene reactive moiety of the maleimide or vinyl sulfone functional groups is capable of undergoing a click chemical reaction with thiol groups of the modified gelatin to form thioether connecting groups between the crosslinker and the modified gelatins.

In certain preferred combination: the exposed reactive moiety of the gelatin is an amine, carbonyl group or thiol, preferably an amine or a carbonyl group; the complementary reactive moiety of the crosslinkable group is a carbonyl group, epoxide, thioimidate, isocyanate isothiocyanate, hydroxyl, amine, thiol, alkene or alkyne, preferably, preferably a thioimidate or an amine; the crosslinkable group is intended to undergo a classical click chemical reaction wherein the reactive moiety in the click chemical reaction is selected from the group consisting of an alkyne, amine, alkene, conjugated diene, thiol, isonitrile and tetrazine, preferably an alkene or a thiol; the crosslinker has the formula Core-(Spacer-R) n, wherein n is an integer of from 2 to 8, preferably 4, wherein Core is C(—CH₂O—)₄, wherein Spacer is (—CH₂CH₂O—)ₘ, m being an integer of from 2 to 150, such as 2 to 50, and wherein the crosslinker is PEG-4MAL or PEG-4VS.

A cross-linked modified gelatin may thus be produced by crosslinking of the crosslinker with the reacting a modified gelatin. The application of water during or after the crosslinking step may thus produce a hydrogel.

The hydrogel may be characterizable by one or more properties, which may be a cross-linked modified gelatin content, a swelling ratio, an equilibrium water content, a crosslinking time and a sol-gel transition temperature, as described in the following.

The hydrogel may be characterizable by a crosslinked modified gelatin content in the range of from 1 to 25% w/v.

The hydrogel may be characterizable by a relaxed mass swelling ratio, wherein the hydrogel of 2.5% wt/v or greater has a relaxed mass swelling ratio of 15 or less, preferably of 14 or 13 or less, and preferably of 12 or less. The hydrogel may also be characterizable by a relaxed mass swelling ratio, wherein the hydrogel of 5% wt/v or greater has a relaxed mass swelling ratio of 9 or less, preferably of 8 or 7 or less, and preferably of 6 or less. The hydrogel may also be characterizable by a relaxed mass swelling ratio, wherein the hydrogel of 10% wt/v or greater has a relaxed mass swelling ratio of 8 or less, preferably of 7 or 6 or less, and preferably of 5 or less. These relaxed mass swelling ratios, including in combination, are preferred when the crosslinkable group is formed using Traut's reagent and the reactive moiety is a thiol, especially when the native gelatin is first functionalized with ethylenediamine, and where the crosslinker is PEG-4MAL.

The Examples below describe a method for measuring relaxed mass swelling ratio, which represents the preferred method.

The hydrogel may be characterizable by an equilibrium swelling ratio, wherein the hydrogel of between 2.5% and 10% wt/v has an equilibrium swelling ratio of from 10 to 25, preferably of from 11 to 24 or of from 12 to 23, and preferably of from 13 to 22 or of from 14 to 21, and most preferably of from 15 to 20. This equilibrium swelling ratio is preferred when the crosslinkable group is formed using Traut's reagent and the reactive moiety is a thiol, especially when the native gelatin is first functionalized with ethylenediamine, and where the crosslinker is PEG-4MAL.

The Examples below describe a method for measuring equilibrium swelling ratio, which represents the preferred method.

The hydrogel may be characterizable by an equilibrium water content, wherein the hydrogel of between 2.5% and 10% wt/v has an equilibrium water content of from 80% to 100%, preferably of 85% to 100%, and preferably of about 90%, or in other words of from 88% to 92%. This equilibrium water content is preferred when the crosslinkable group is formed using Traut's reagent and the reactive moiety is a thiol, especially when the native gelatin is first functionalized with ethylenediamine, and where the crosslinker is PEG-4MAL.

The Examples below describe a method for measuring equilibrium swelling ratio, which represents the preferred method.

The hydrogel may be characterizable by a crosslinking time, wherein the hydrogel of between 2.5% and 10% wt/v has a crosslinking time, at room temperature, of from about 1 second to about 5 seconds, preferably from about 1 second to about 4 seconds, or from about 1 second to about 3 seconds, and preferably about 1.2 or about 1.8 seconds, or in other words of from 1 second to 2 seconds. This crosslinking time is preferred when the crosslinkable reactive moiety is a thiol, especially when the native gelatin is first functionalized with L-cysteine, and where the crosslinker is PEG-4MAL.

The Examples below describe a method for measuring crosslinking time, which represents the preferred method.

The hydrogel may be characterizable by a crosslinking time. For example, in some embodiments, the hydrogel of between 2.5% and 10% wt/v may be prepared from a system (i.e. modified gelatin and crosslinker) which has a crosslinking time, at room temperature (e.g. at 25° C.), of from about 1 second to 1 minute, or from about 1 second to about 30 seconds, or from about 1 second to about 10 seconds, or from about 10 seconds to about 30 seconds, or from about 20 seconds to about 30 seconds. Such crosslinking times may for example be achievable in some embodiments when the crosslinkable reactive moiety is a thiol (for example when the native gelatin is first functionalized with L-cysteine), and where the crosslinker is PEG4-MAL.

In some other embodiments, the hydrogel of between 2.5% and 10% wt/v may be prepared from a system which has a crosslinking time, at room temperature (e.g. at 25° C.), of from about 1 minute to about 5 minutes, or from about 5 minutes to 10 minutes, or from about 10 minutes to about 30 minutes, or from about 20 minutes to about 30 minutes, or from about 22 minutes to about 28 minutes, or from about 25 minutes to about 27 minutes. Such crosslinking times may for example be achievable in some embodiments when the crosslinkable reactive moiety is a thiol, especially when the native gelatin is first functionalized with L-cysteine, and where the crosslinker is PEG-4VS.

The Examples below describe a method for measuring crosslinking time, which represents the preferred method.

### Kits

Also provided is a kit for producing a crosslinked modified gelatin. The kit may contain a native gelatin derived from a marine source and one or more crosslinkable group species for producing a modified gelatin, as described herein. The kit may alternatively or in addition contain a modified gelatin and optionally a crosslinker for producing a cross-linked modified gelatin as described herein.

In preferred embodiments, the kit contains a modified gelatin as described herein, and a crosslinker as described herein.

In other preferred embodiments, the kit contains a native gelatin derived from a marine source, and a crosslinkable group species for producing a modified gelatin as described herein, optionally along with a crosslinker for producing a cross-linked modified gelatin as described herein.

The kit may also contain water for producing a hydrogel as described herein. The kit may for example contain a buffer, such as HEPES. For example, the kit may contain an aqueous solution of a buffer, such as HEPES. The kit may also contain instructions for producing one or more of a modified gelatin, a crosslinked gelatin, and a hydrogel, as described herein.

### Uses of Crosslinked Gelatins and Hydrogels

Crosslinked modified gelatins and hydrogels as described herein have utility as biomaterials, and particularly as cell growth/storage matrices and for 3D bioprinting.

In particular, the present inventors have found that cell growth matrices comprising hydrogels of the present disclosure are essentially equally useful for growing, storing and maintaining encapsulated cells, under growth conditions (in growth media, 37° C., 5% CO₂), in a viable state, as hydrogels produced from photoinitiator crosslinked methacryloyl-functionalized gelatins in the short-term (up to 2 weeks), and actually more effective in the long term (2 weeks onwards).

In preferred embodiments, that cell growth matrix consists of a hydrogel as herein described (growth media notwithstanding).

Cells may be encapsulated by forming the hydrogel around a cell population. This may involve providing a cell population, suspending the cell population in an aqueous solution of modified gelatin as herein described, and mixing in a crosslinker as herein described under, or followed by subjecting the suspension to, conditions suitable to effect the crosslinking reaction.

The Examples below describe a method for encapsulating cells, which represents the preferred method.

In preferred embodiments, a population of cells encapsulated under growth conditions in a hydrogel formed from an aqueous solution of modified gelatin of from 2.5% wt/v to 10% wt/v are at least 82% viable, preferably 83% viable and preferably 84% viable, after 1 day, preferably 7 days, preferably 14 days and preferably 21 days. Preferably, a population of cells encapsulated in a hydrogel formed from an aqueous solution of modified gelatin of from 2.5% wt/v to 5% wt/v are at least 84% viable, preferably 85% viable, after 1 day, preferably 7 days, and preferably 14 days, and preferably at least 86% viable are after 1 day, preferably 7 days. Preferably a population of cells encapsulated in a hydrogel formed from an aqueous solution of modified gelatin of about 2.5% wt/v are at least 86% viable after 1 day, preferably 7 days. More preferably, after 21 days, a population of cells encapsulated in a hydrogel formed from an aqueous solution of modified gelatin of 5% wt/v to 10% wt/v are at least about 84% viable, and a population of cells encapsulated in a hydrogel formed from an aqueous solution of modified gelatin of about 2.5% wt/v are at least about 85%, preferably 86%, viable.

The Examples below describe a method for measuring cell viability, which represents the preferred method.

In preferred embodiments, a population of cells encapsulated under growth conditions in a hydrogel formed from an aqueous solution of modified gelatin of from 2.5% wt/v to 10% wt/v have a metabolic activity (AU) after 7 days of from about 400 AU to about 1000 AU, preferably from about 450 AU to about 800 AU, preferably from about 500 AU to about 600 AU. In some embodiments, the hydrogel is formed from a modified gelatin and PEG-4MAL. In other embodiments, the hydrogel is formed from a modified gelatin and PEG-4VS.

The Examples below describe a method for measuring metabolic activity, which represents the preferred method.

In preferred embodiments, the cells are encapsulated in a concentration of about 2×10⁶ cells/mL, or in other words the cell population comprises cells in numbers of about 2×10⁶ cells/mL. In other preferred embodiments, the cells are encapsulated in a concentration of about 1×10⁶ cells/mL, or in other words the cell population comprises cells in numbers of about 1×10⁶ cells/mL.

In preferred embodiments, the cells are mammalian cells, preferably human cells, and preferably human cancerous cells. Breast cancer cells are the representative embodiment.

### EXAMPLES

### Example 1

### Synthesis of an Exemplary Modified Gelatin: Thiolated Cold-Water Fish Skin Gelatin Via EDC/NHS Coupling of L-Cysteine

10 g gelatin from cold-water fish skin (MW~60 kDa, Sigma®, Lot #SLCG7135) was added to 500 mL of 0.1 mM HCl and stirred at room temperature until dissolved. Then, 7.5 g 1-ethyl-3 (3-dimethylamino) propyl carbodiimide (EDC) (Sigma®) and 3.75 g N-hydroxysuccinimide (NHS) (Sigma®) were added to the solution. The EDC/NHS reaction was allowed to continue for 30 minutes, then 20 g L-cysteine (Sigma®) was added to the solution. The conjugation reaction proceeded for 24 hours at room temperature protected from light. The pH of the solution was maintained at 5.0 throughout the reaction. Then, the solution was dialysed against 0.1 mM HCl using 1 kDa molecular weight cut-off snakeskin dialysis tubing (Sigma®) for 5 days. Once dialysis was complete, samples were frozen overnight at −80° C. and lyophilised for 5 days.

### Example 2

### Synthesis of an Exemplary Modified Gelatin: Amination of Cold-Water Fish Skin Gelatin

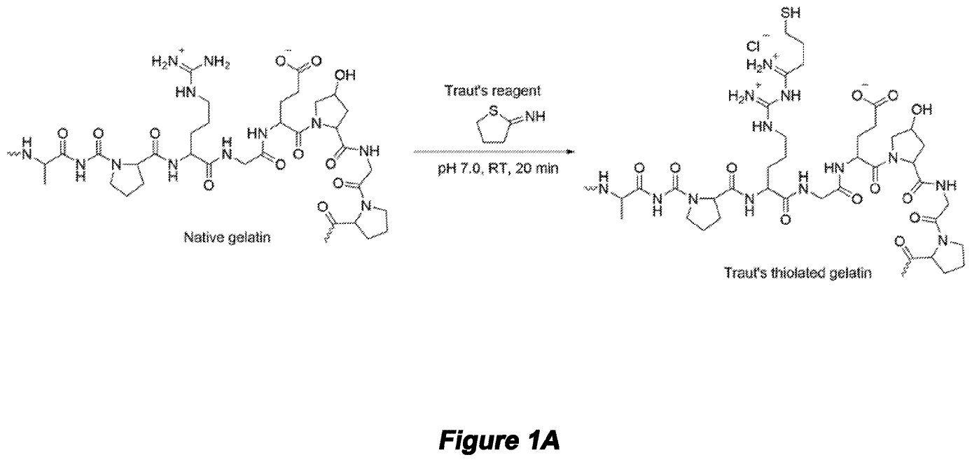

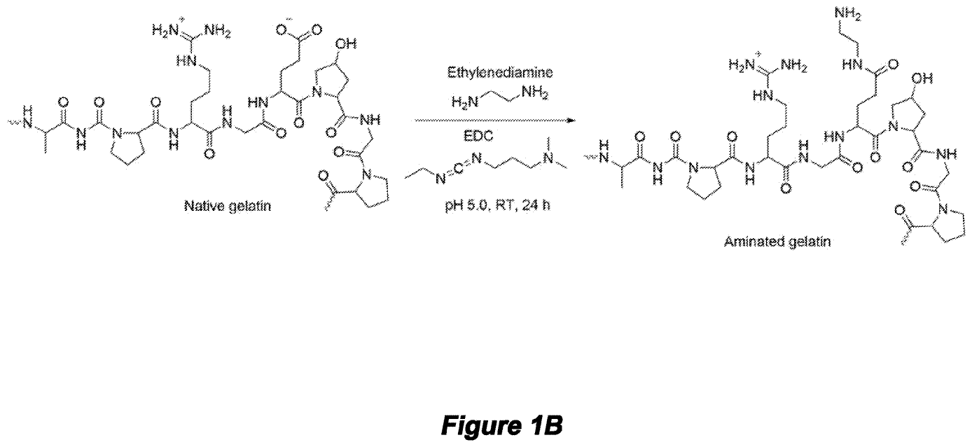

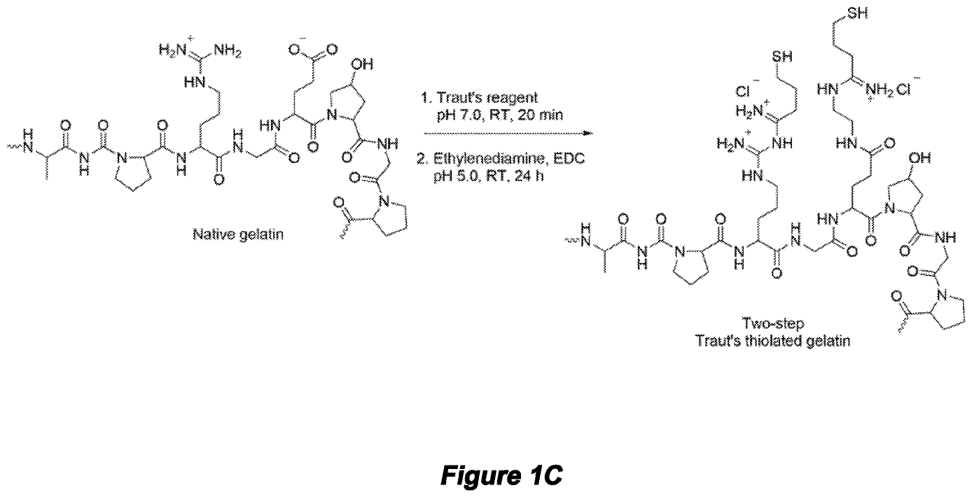

Gelatin from cold-water fish skin was aminated via the following method: 6 g gelatin from cold-water fish skin was dissolved in 150 mL 0.1 M phosphate-buffered saline (PBS) (Sigma-Aldrich®, St. Louis). Then, 60 g ethylenediamine (Sigma-Aldrich®, St. Louis) was added to the solution. The pH of the solution was adjusted to 5.0 using HCl and NaOH and 2.3 g EDC was added. The reaction was left stirring using a magnetic stir bar for 24 hours at room temperature protected from light. Once the reaction time had lapsed, samples were dialysed, frozen and lyophilized. The reaction scheme is shown in FIG. 1B. Aminated gelatin was thiolated using Traut's reagent as follows.

### Synthesis of an Exemplary Modified Gelatin: Thiolated Gelatin Using Traut's Reagent

Thiolation of gelatin using Traut's reagent was adapted from the protocol originally described by Duggan et al. 1 g of gelatin from cold-water fish skin was added to 100 mL ultrapure water and dissolved under stirring at room temperature. Once dissolved, the pH of the solution was adjusted to 7.0 and a 2-fold molar excess of 2-iminothiolane (Traut's reagent) (Sigma®) was then added to the solution. The reaction was left stirring for 24 hours at room temperature and protected from light. Then, the solution was dialysed against 0.1 mM HCl using 1 kDa molecular weight cut-off snakeskin dialysis tubing (Sigma®) for 5 days. Once dialysis was complete, samples were frozen overnight at −80° C. and lyophilised for 5 days. The reaction scheme is shown in FIGS. 1A and 1C.

### Example 3

### Determination of Amine Content in Gelatin and Gelatin Derivatives

The amine content of gelatin and gelatin derivatives was quantified via 2,4,6-trinitrobenzenesulfonate (TNBS) assay, as described by Meinert et al. Briefly, 0.1 M NaHCO₃ buffer was prepared, and the pH of the solution was adjusted to 8.5 using HCl and NaOH. A 0.01% (wt/v) TNBS solution was prepared by 1:500 dilution of TNBS stock. Gel-SH and gelatin from cold-water fish skin were then dissolved in 0.1 M NaHCO₃ buffer at 10 mg/mL. 250 μL of each solution was diluted to 500 g/mL using 0.1 M NaHCO₃ buffer. A 1:2 dilution series of Gel-SH and gelatin from cold-water fish skin was prepared with concentrations ranging from 0-500 μg/mL. An L-Cysteine standard dilution series of 0.5 mM-0.156 mM was prepared. 200 μL of each sample and standard dilution was added in triplicate to a clear 96-well plate (Corning® Costar®), and 100 μL 0.01% (wt/v) TNBS solution was added. Samples were then mixed on a plate shaker for 5 minutes protected from light. Then, samples were transferred to a 37° C. laboratory oven and incubated for 2 hours protected from light. Well-plate absorbance was read at 335 nm using a CLARIOstar® spectrophotometer. The amine content of the samples was determined through comparison of sample absorbance to the absorbance of the L-Cysteine standard curve.

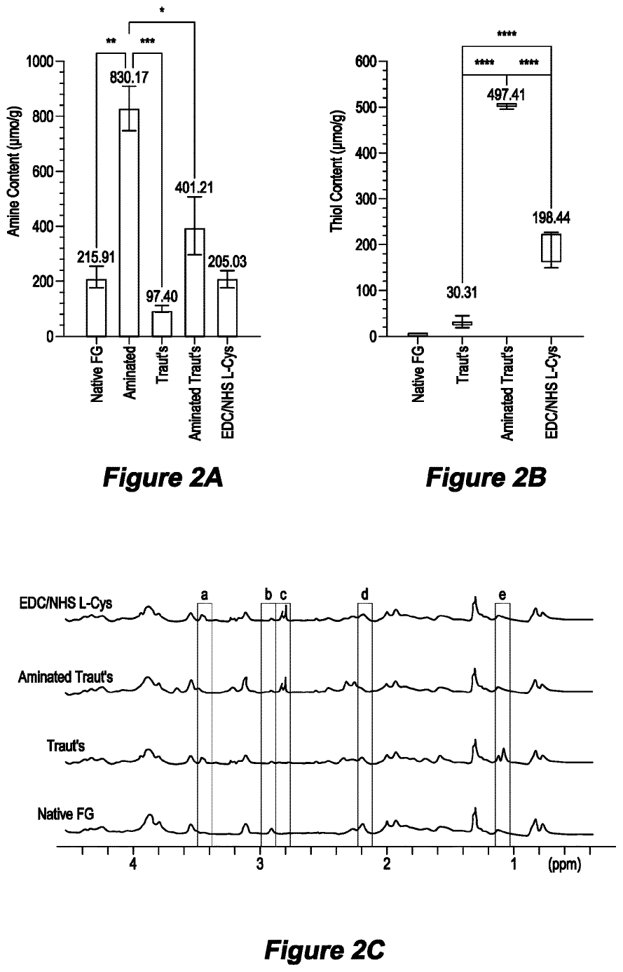

FIG. 2A shows that the amine content was increased almost 4-fold in cold-water adapted fish gelatin aminated using ethylenediamine (830.17 μmol/g) as compared with native (not aminated) fish gelatin (215.91 μmol/g). The figure further shows that the amine content of this aminated gelatin is reduced when treated with Traut's reagent (401.21 μmol/g). The amine content of native gelatin is also reduced when treated with Traut's reagent (97.40 μmol/g), and the amine content of native gelatin treated with EDC/NHS coupling of L-cysteine is similar (205.03 μmol/g) to native gelatin.

### Quantification of the Thiol Content in Gelatin and Gelatin Derivatives

The thiol content of native and thiolated cold-water adapted fish gelatin (Gel-SH) was quantified via 5,5′-Dithio-bis-(2-nitrobenzenoic acid) (DTNB) assay, as previously described by Deng et al. A 1:2 L-Cysteine standard dilution series of 0-2 mM was prepared. Gel-SH and gelatin from cold-water fish skin were dissolved at 5 mg/mL in PBE. A 1:2 dilution series of samples was prepared with concentrations ranging from 500 μg/mL-125 μg/mL. 25 μL of each sample and standard was then added in triplicate to a clear 96-well plate (Corning® Costar®). 125 μL of DTNB solution was then added to each sample. The well plate was then shaken on a plate shaker and incubated at room temperature protected from light for 15 minutes. Post-incubation, the absorbance of the well-plate was measured at 412 nm using a CLARIOstar® well-plate reader. The thiol content of Gel-SH was determined through comparison of sample absorbance to the absorbance of the L-Cysteine standard curve.

FIG. 2B shows that the thiol content was increased in native cold-water adapted fish gelatin when reacted with Traut's reagent (30.31 μmol/g) as compared with the native gelatin (approximately baseline). Thiol content was further increased in aminated (using ethylenediamine) native cold-water adapted fish gelatin treated with Traut's reagent (497.41 μmol/g). Thiol content was also increased in native gelatin treated with EDC/NHS coupling of L-cysteine (198.44 μmol/g) as compared to native gelatin.

### ¹H-NMR

Proton nuclear magnetic resonance (1H-NMR) was conducted to characterise the molecular profile of Gel-SH. Gel-SH and gelatin from cold-water fish skin were dissolved in 90% H₂O/10% D₂O to a final concentration of 1% (wt/v). 1 mL of each sample was added to respective NMR tubes and sample spectra were collected using a Bruker Avance 600 MHz NMR instrument with water suppression. The sample spectra were analyzed using Bruker TopSpin 3.6.4. FIG. 2C contains comparative spectra.

### Rheology

The rheological properties of thiolated cold-water fish skin gelatin (Gel-SH) and porcine skin gelatin (Type A, 300 bloom, Sigma-Aldrich) were determined using an Anton-Paar modular compact rheometer (MCR) 302. Shear-rate sweeps were conducted at 25° C. using a 25 mm cone plate (CP25), with shear-rate range of 0.1-1,000/s, at a constant frequency of 1 Hz. Temperature sweeps were conducted using a 25 mm parallel plate (PP25) at a constant frequency of 1 Hz, and constant strain of 1%, with temperature ramping linearly from 40° C.-0° C., at a rate of 2° C./min.

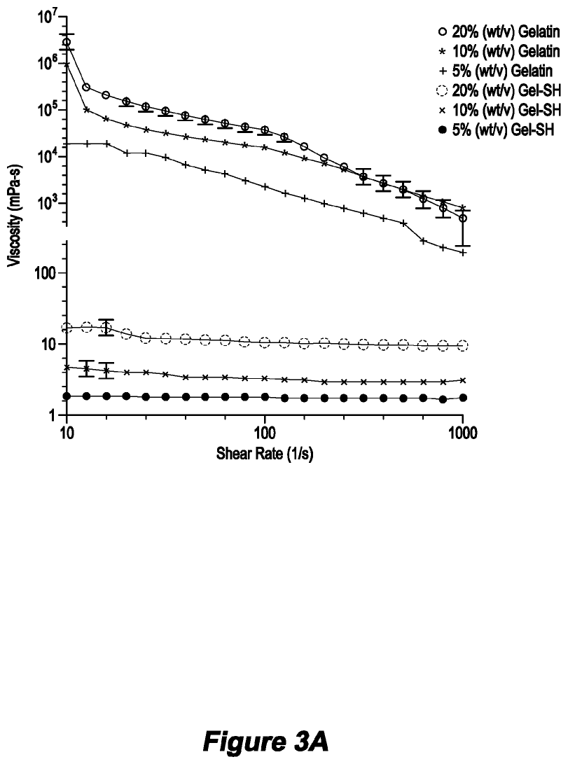

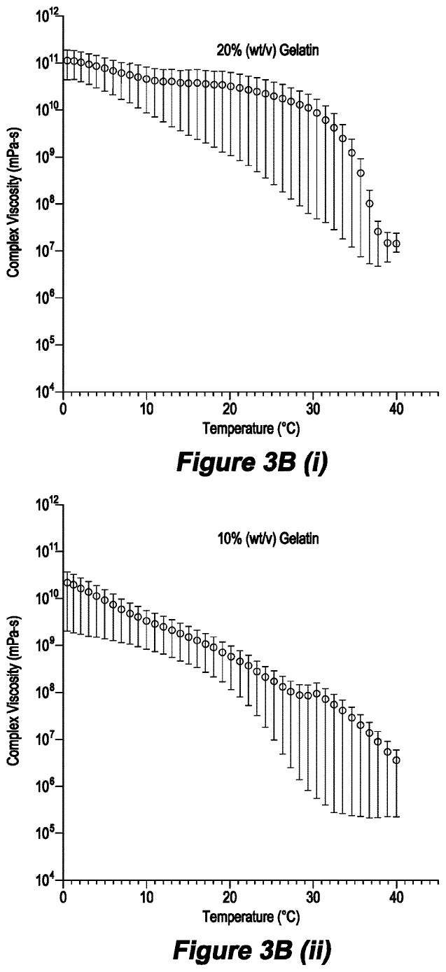

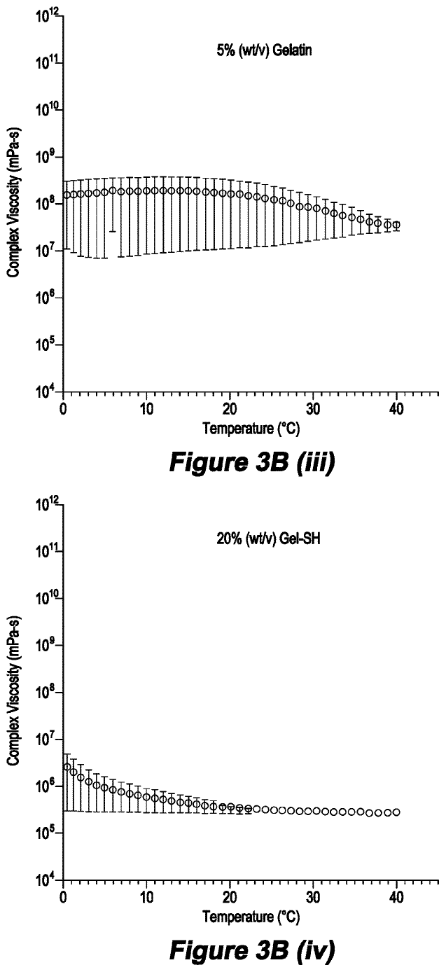

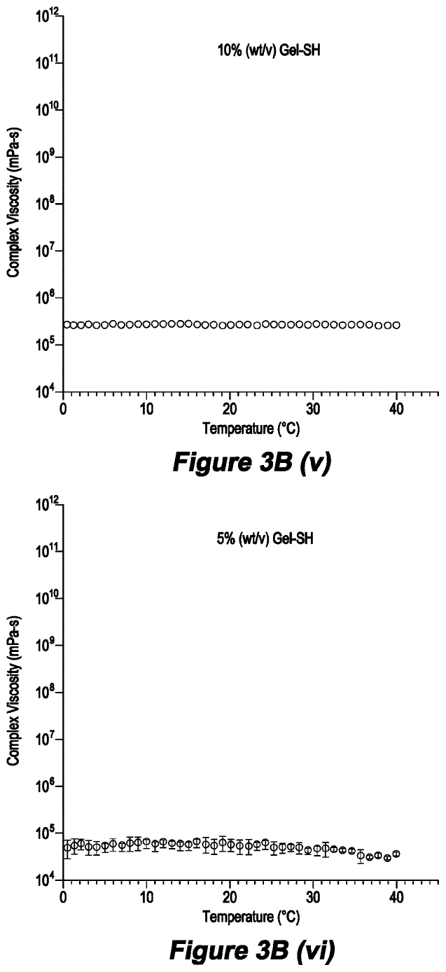

FIG. 3 demonstrates lower viscosity and reduced temperature dependence of thiolated cold-water fish skin gelatin solutions compared to solutions of mammalian (porcine) gelatin, enabling easier liquid handling and improved volumetric reproducibility.

### Example 4

### Crosslinking of Modified Gelatins Via a Click Chemical Reaction: Gel-SH/PEG-4MAL Hydrogel Preparation

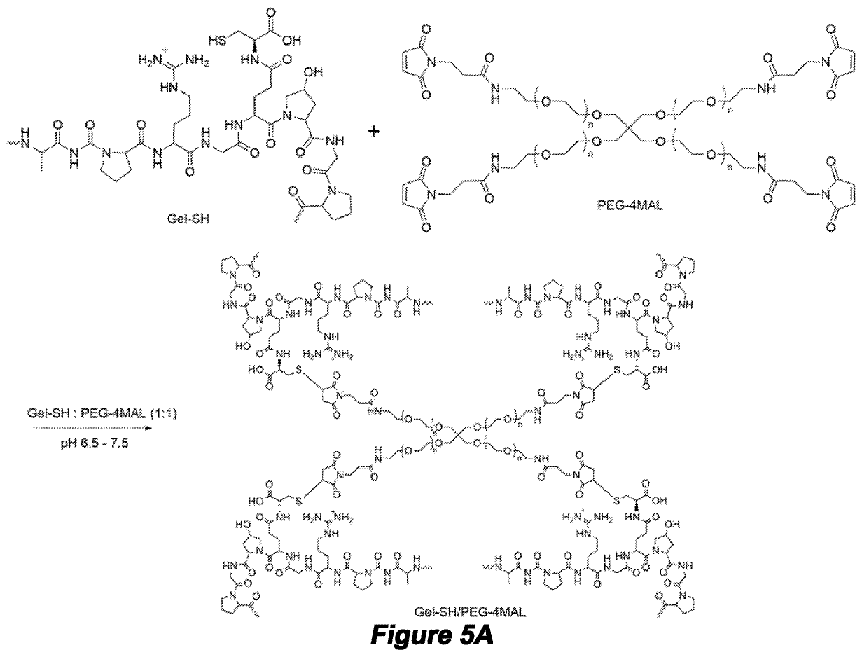

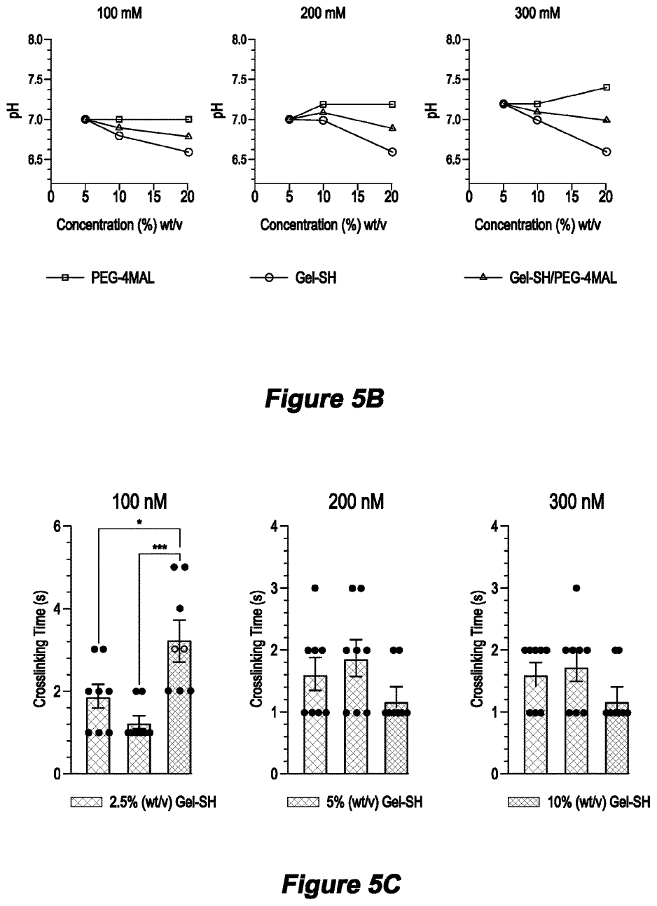

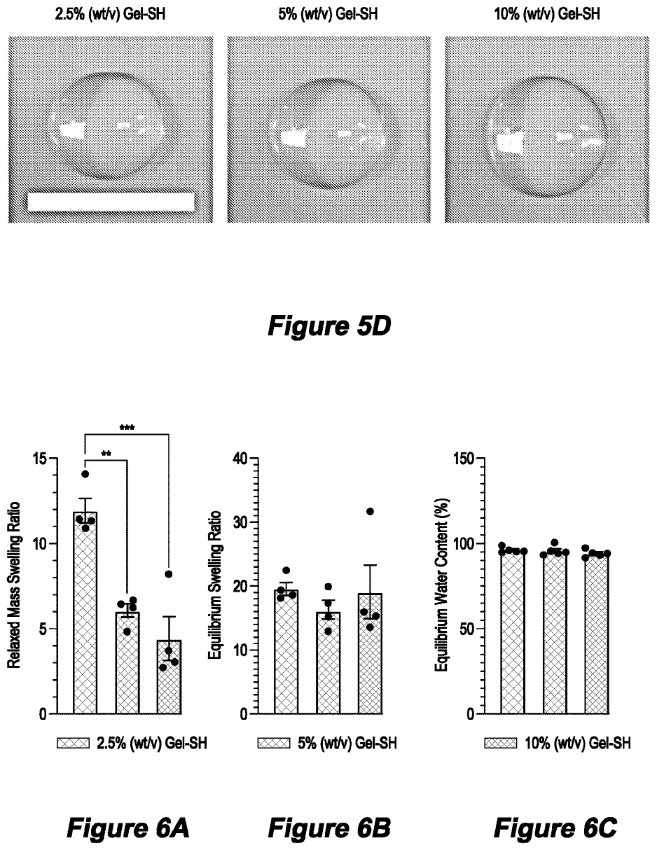

Gel-SH and PEG-4MAL (MW 20 kDa, JenKem®) were dissolved in 300 mM, 200 mM or 100 mM (4-2-hydroxyethyl)-1-piperazineethanesulfonic acid (HEPES) buffer (Gibco™, Lot #2185833), at 20% (wt/v), 10% (wt/v) or 5% (wt/v), respectively. Various concentrations of HEPES buffer were used to prepare Gel-SH, PEG-4MAL and Gel-SH/PEG-4MAL hydrogels, to determine the effect of HEPES buffer concentration on hydrogel precursor pH and hydrogel crosslinking times. FIG. 5B shows the pH of the hydrogel precursor solutions (PEG-4MAL and Gel-SH) prepared in 100 mM, 200 mM, or 300 mM HEPES, respectively, at concentrations ranging from 5-20% (wt/v). The precursor solutions were mixed in equimolar amounts (based on the number of thiol groups in Gel-SH, and the number of maleimide groups in the crosslinker PEG-4MAL), under ambient light conditions, crosslinked modified gelatins were produced. The pH of each hydrogel precursor and hydrogel was determined using test strips. To prepare Gel-SH/PEG-4MAL hydrogels with 10% (wt/v) final Gel-SH concentration, 20% (wt/v) Gel-SH precursor solution was mixed with 20% (wt/v) PEG-4MAL precursor solution in an equimolar amount (based on the number of thiol groups in Gel-SH and the number of maleimide groups in PEG-4MAL) in 96-well plates (Corning®). The reaction scheme is shown in FIG. 5A. Crosslinking time was determined via pipette mixing, where the time of crosslinking was defined as the time at which hydrogel solution could no longer be pipetted. FIG. 5C shows the crosslinking time following mixing of the hydrogel precursor solutions (PEG-4MAL and Gel-SH) prepared in either 100 mM, 200 mM, or 300 mM HEPES, respectively, at final Gel-SH concentrations ranging from 2.5-10% (wt/v). 300 mM HEPES enabled the pH of the combined hydrogel precursor solutions (Gel-SH/PEG-4MAL) to remain between 7-7.5, which is as suitable pH range for highly stereoselective click chemistry reaction and compatible with cells.

### Mechanical Testing

Gel-SH/PEG-4MAL hydrogels with 10% (wt/v), 5% (wt/v) and 2.5% (wt/v) final Gel-SH concentration and equimolar amounts of PEG-4MAL, respectively, were prepared in 300 mM HEPES buffer and allowed to swell overnight in phosphate-buffered saline (PBS) at 37° C. Prior to compression testing, hydrogels were imaged using a Nikon® SMZ25 stereomicroscope, and the surface area was determined. Hydrogels were then submerged in a PBS-filled water bath at 37° C. and compressed in an unconfined configuration using an Instron 5567 (Instron) equipped with a 5 N load cell and non-porous aluminum indenter at a strain rate of 0.01 mm/s. The compressive Young's modulus E of the hydrogels was determined as the slope of stress-strain curve at 0.1-0.15 mm/mm strain as described by Kahl et al.

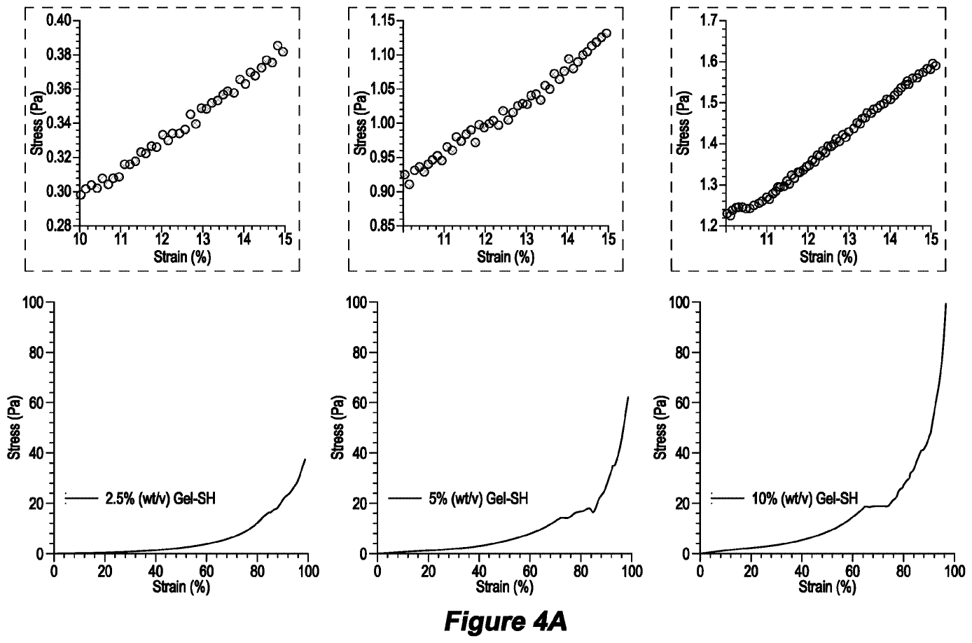

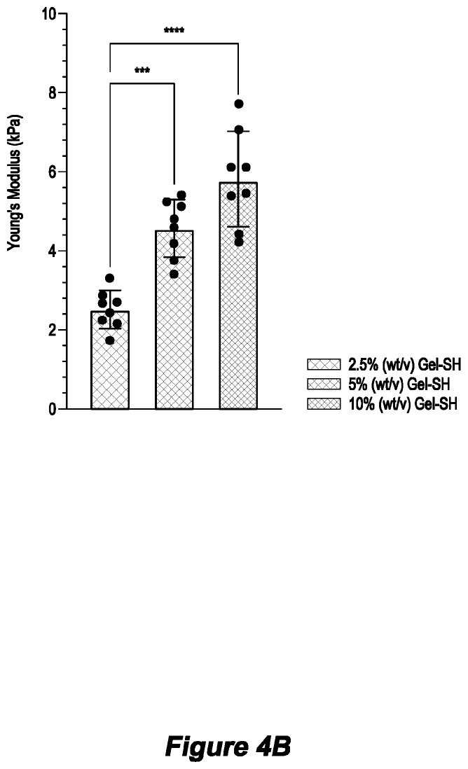

FIG. 4A shows representative stress-strain curves and FIG. 4B shows the Young's moduli obtained for hydrogels with final Gel-SH concentration of 2.5-10% (w/v) and demonstrates a concentration-dependent increase in mechanical properties of the crosslinked hydrogel.

### Equilibrium Swelling and Mass Swelling Ratio

Hydrogels with 10% (wt/v), 5% (wt/v) and 2.5% (wt/v) Gel-SH and equimolar PEG-4MAL concentration were prepared using 300 mM HEPES buffer and weighed immediately post-crosslinking. Hydrogels were then allowed to swell overnight in PBS at 37° C. overnight. After swelling, hydrogels were weighed again, then lyophilized. The recorded weights of the hydrogels were used to calculate the equilibrium water content (EWC) using the following equation:

$$EWC ( \% ) = \frac{( {m}_{wet} - {m}_{lyophilized} )}{{m}_{w e t}} \times 100$$

where m_{wet} is the mass of hydrogel post-swelling, and m_{lyophilised} is the mass of hydrogel after lyophilization.

The equilibrium mass swelling ratio (Qₘ) was calculated using the following equation:

$${Q}_{m =} \frac{( {m}_{wet} - {m}_{lyophilized} )}{{m}_{wet}}$$

The relaxed mass swelling ratio (Qₘᵣ) was determined using the following equation:

$${Q}_{m r =} \frac{( {m}_{crosslinked} - {m}_{lyophilized} )}{{m}_{crosslinked}}$$

where m_{crosslinked} is the mass of hydrogel immediately post-crosslinking.

Relaxed mass swelling ratio (Qₘᵣ) describes the relationship between the weight of the dried hydrogel, and the weight of the hydrogel immediately after crosslinking. The equilibrium swelling ratio (Qₘ) describes the relationship between the weight of the dried hydrogel and the weight of the hydrogel after swelling. Equilibrium water content (EWC) describes the capacity for the hydrogel to retain water when the osmotic and ionic pressure of solutions external to the hydrogel matrix are at equilibrium with the pressure of the hydrogel matrix.

FIG. 6A shows a negative correlation between hydrogel concentration and the relaxed mass swelling ratio. FIG. 6B and FIG. 6C demonstrate that equilibrium swelling ratio and the equilibrium water content, respectively, are not influenced by the hydrogel concentration within the range tested.

### Example 5

### Cell Encapsulation in Gel-SH/PEG-4MAL Hydrogels

Stock solutions of 20% (wt/v), 10% (wt/v), and 5% (wt/v) Gel-SH and PEG-4MAL were prepared in 300 mM HEPES buffer. Cells were lifted using 0.25% Trypsin/ethylenediaminetetraacetic acid (EDTA) and counted. MCF-7 breast cancers were resuspended in Gel-SH at a concentration of 2×10⁶ cells/mL. A volume of 10 μL PEG-4MAL solution was added to 48-well plates (Corning®), then, an amount of Gel-SH cell suspension providing an equimolar amount of Gel-SH (based on the number of thiol groups in Gel-SH, and the number of maleimide groups in the crosslinker PEG-4MAL) was pipette mixed with PEG-4MAL solution until crosslinking occurred. Post-crosslinking, cell-laden hydrogels were incubated in RPMI 1640 medium (Gibco™) supplemented with 10% (v/v) foetal bovine serum (FBS), 1% (v/v) P/S, 1% (v/v) non-essential amino acids, 1% (v/v) sodium pyruvate, and 0.1% (v/v) insulin-transferring-selenium (all ThermoFisher™) at 37° C. in a humidified cell incubator with 5% CO₂.

### Cell Viability

The viability of cells encapsulated in Gel-SH/PEG-4MAL hydrogels was determined using fluorescein diacetate (FDA) (ThermoFisher™)/propidium iodide (PI) (ThermoFisher™) assay. Cell media was aspirated, and samples were washed with PBS at room temperature for 5 minutes, then incubated with staining solution (10 μg/mL FDA and 5 μg/mL PI in PBS) for 2 minutes. The staining solution was aspirated, and samples were washed for 2 minutes in PBS. The samples were then transferred to a glass slide and imaged using either a Leica SP5 confocal microscope or Nikon® SMZ25 epifluorescence microscope. Z-stacks of hydrogels were captured with 10 μm slice intervals, and maximum intensity projections of hydrogel Z-stacks were obtained using ImageJ. Cell viability was determined through quantification of particles in live and dead channels of maximum intensity projections.

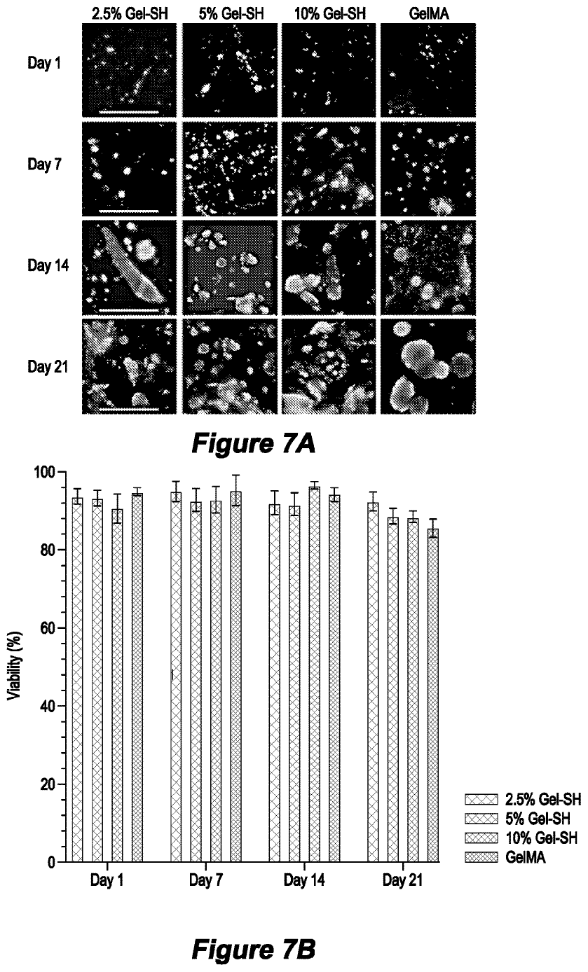

FIG. 7A shows representative live/dead images for cells encapsulated Gel-SH/PEG-4MAL hydrogels with final Gel-SH concentrations ranging from 2.5-10% (wt/v) Gel-SH, and photocrosslinkable gelatin methacryloyl (GelMA; 5% w/v) control hydrogels. FIG. 7B shows that high cell viabilities are maintained over a culture period of 21 days, demonstrating cytocompatibility of the GelSH-based hydrogels.

### Nuclei and F-Actin Staining

Staining of the nuclei and f-actin filaments of MCF-7 cells encapsulated in Gel-SH/PEG-4MAL hydrogels was conducted using diamidino-2-phenylindole (DAPI) (ThermoFisher™/Alexa-Fluor™ 488-conjugated phalloidin stains. On day 1, 7, 14 and 21, media was removed from MCF-7 hydrogel wells. Hydrogels were then washed with 1 mL PBS for 10 minutes at RT. Then, PBS was aspirated, and samples were fixed in 1 mL 4% (wt/v) paraformaldehyde (PFA) for 1 hour. Post-fixing, PFA was aspirated, and hydrogels were washed with 1 mL PBS. PBS for washing was aspirated, and an additional 1 mL aliquot of PBS was added to the hydrogels. Hydrogels were then stored at 4° C. PBS was aspirated from wells and hydrogels were blocked using 300 μL blocking buffer (5% (v/v) goat serum (Gibco™), 0.1% (v/v) Triton X-100 (Sigma-Aldrich™) per hydrogel overnight on plate shaker at 4° C. The blocking buffer was then aspirated, and hydrogels were washed twice with PBS at RT for 5 minutes each wash. PBS was aspirated and 150 μL of 1:1,000 DAPI, 1:200 phalloidin in PBS was added to each hydrogel. The hydrogels were incubated at 4° C. overnight on plate shaker. Post-incubation, the staining solution was aspirated, and hydrogels were washed with 300 μL washing buffer (20% (v/v) blocking buffer, 1% (v/v) goat serum) per hydrogel three times over the course of 8 hours at 4° C. on plate shaker. Post-washing, washing buffer was aspirated and hydrogels were washed with PBS three times and stored at 4° C. until imaging. The hydrogels were imaged using Leica SP5 confocal microscope at 4× objective. The DAPI channel was captured at an excitation wavelength of 405 nm, and phalloidin was captured at an excitation wavelength of 488 nm.

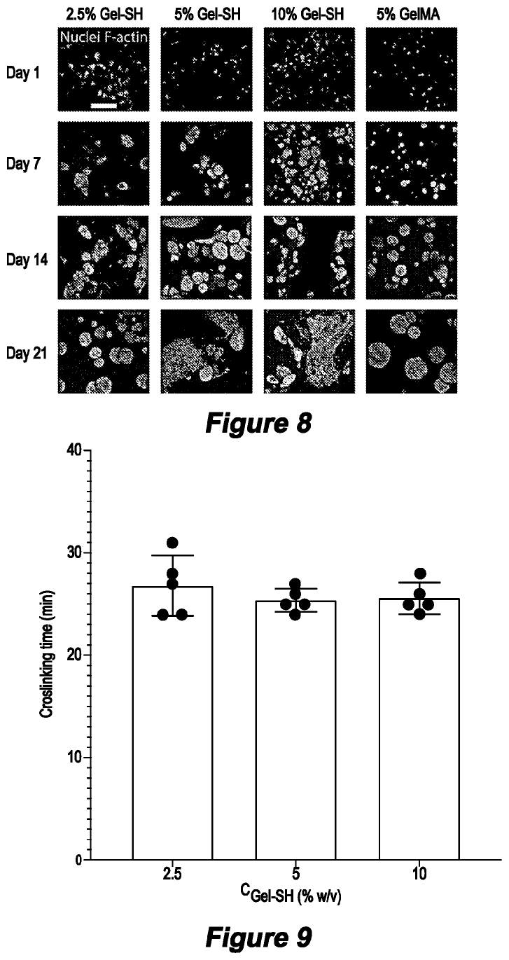

FIG. 8 shows that Gel-SH/PEG-4MAL based hydrogels with final Gel-SH concentrations ranging from 2.5-10% (wt/v) support the formation multicellular spheroids similar to GelMA controls, which are regarded as a gold standard gelatin derivative in 3D cell culture applications.

### Example 6

### Crosslinking Time of Gel-SH/PEG-4MAL Hydrogels Versus Gel-SH/PEG-4VS Hydrogels

Hydrogels were formed by reaction of Gel-SH with 4-arm PEG-maleimide (PEG-4MAL, MW 20 kDa, JenKem®) and Gel-SH with 4-arm PEG-vinyl sulfone (PEG-4VS, MW 20 kDa, JenKem®), respectively, to assess differences in crosslinking duration between the two PEG-based activators, as outlined in Example 4.

For PEG-4MAL-based reactions, both Gel-SH and PEG-4MAL were dissolved in 300 mM HEPES buffer (Gibco™, Lot #2185833) at 20% (wt/v), respectively. For PEG-4VS-based reactions, Gel-SH and PEG-4VS were both dissolved in 300 mM HEPES containing 300 mM triethanolamine (TEA, Sigma-Aldrich), respectively. Gel-SH and PEG-based activator solutions were mixed in an equimolar ratio (based on the number of thiol groups in Gel-SH, and the number of maleimide groups in the crosslinker PEG-4MAL, or the number of vinyl sulfone groups in the crosslinker PEG-4VS) to create hydrogels with a final Gel-SH concentration of 2.5%, 5%, and 10% w/v, respectively. The crosslinking time at room temperature was determined as the time at which the hydrogel precursor solution could no longer be aspirated into a pipette due to gel formation. The results for Gel-SH crosslinked with PEG-4MAL are shown in FIG. 5 C, while results for Gel-SH crosslinked with PEG-4VS are shown in FIG. 9.

### Example 7

### Cell Encapsulation in Gel-SH/PEG-4MAL Hydrogel Versus Gel-SH/PEG-4VS Hydrogel

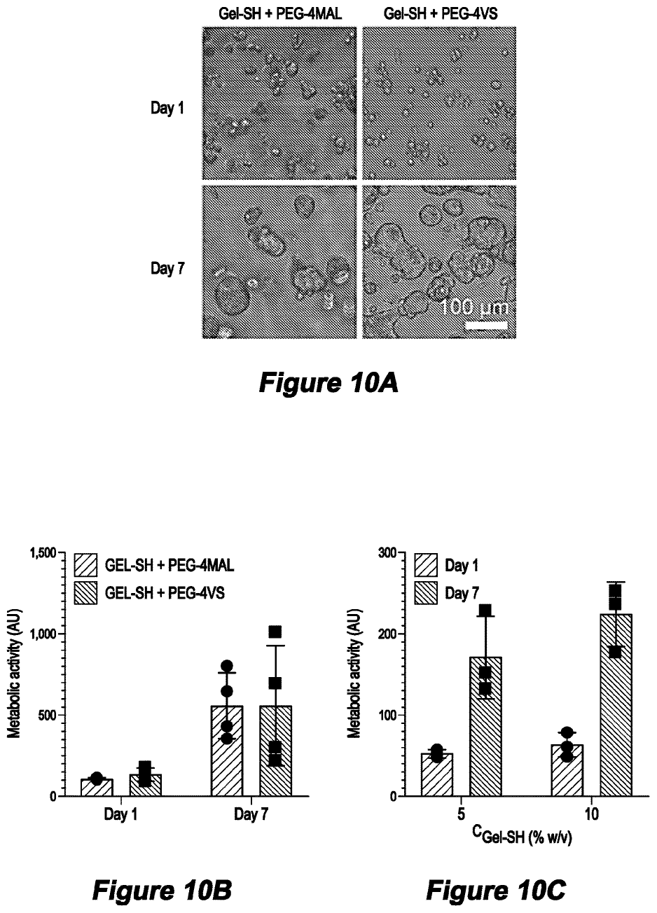

MCF-7 breast cancer cells were encapsulated at a density of 1 million cells/mL hydrogel precursor solution in hydrogels form of 10% (w/v) Gel-SH with PEG-4MAL or PEG-4VS, respectively, prepared as outlined in Example 5. Viability and growth of MCF-7 cells was assessed using brightfield microscopy using a Nikon Eclipse Ts2 Inverted Microscope (FIG. 10A) and PrestoBlue™ Cell Viability Reagent (ThermoFisher Scientific) (FIG. 10B) following manufacturer's instructions at day 1 and day 7 of culture. Briefly, individual cell-laden hydrogels were incubated at 37° C. and 5% CO₂ in a humidified cell culture incubator (Binder CB 170) with 900 μL cell culture media and 100 μL PrestoBlue™ Cell Viability Reagent for 1 hour. Following the incubation period, samples (100 μL) of the cell culture media containing PrestoBlue™ Cell Viability Reagent were transferred to a Nunc™ Micro Well™ 96-Well plate (ThermoFisher Scientific), and cellular metabolic activity was assessed using fluorescence measurements (excitation wavelength: 560 nm; emission wavelength: 590 nm) in a CLARIOstar® Plus (BMG LabTech) plate reader and expressed as arbitrary units (AU).

In a second experiment, the effect of Gel-SH concentration was assessed. MCF-7 cells were encapsulated at 1 million cells/mL in hydrogels containing 5% or 10% w/v Gel-SH and equimolar amounts of PEG-4VS, as outlined for Example 5, and metabolic activity was assessed at day 1 and day 7 of culture (FIG. 10C) following the methods outlined above.

This data demonstrates that the Gel-SH materials of the present invention can be crosslinked using a variety of Michael-addition/click-reactive moieties. Here it was shown that PEG-vinyl sulfone crosslinks Gel-SH much slower than PEG-maleimide, offering additional time for liquid handling. The slower kinetics of the PEG-vinyl sulfone is particularly useful in high throughput liquid handling where the cell-containing Gel-SH suspension can be pre-mixed with PEG-vinyl sulfone solution in a larger master volume, that may be later dispensed robotically. This further amplifies the benefits of having a low viscosity precursor solution, which itself is also ideal for automated liquid handling. This is in contrast to gels formed with PEG-MAL, which crosslink very quickly and therefore must be used straight immediately, and must be mixed/formed individually.

The above examples are only the preferred examples of the present disclosure. It shall be pointed out that various improvements and modifications could be made by those ordinarily skilled in the art without deviating from the principle of the present disclosure, which shall fall within the protection scope of the present disclosure.

In the claims and in the preceding description, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the disclosure.

### REFERENCES

1. Deng et al., Development of Disulfide Bond Crosslinked Gelatin/ε-Polylysine Active Edible Film with Antibacterial and Antioxidant Activities, Food Bioprocess Technol., 13, 577-588.
2. Duggan et al., Synthesis of Mucoadhesive Thiolated Gelatin Ysing a Two-Step Reaction Process, European Journal of Pharmaceutics and Biopharmaceutics, 2015, 91, 75-81.
3. Kahl et al. MechAnalyze: An Algorithm for Standardization and Automation of Compression Test Analysis, Tissue Engineering Part C: Methods, 2021, 27.
4. Loessner et al, Functionalization, preparation and use of cell-laden gelatin methacryloyl-based hydrogels as modular tissue culture platforms, Nature Protocols, 2016, 11, 727-746.
5. Meinert et al., A Method for Prostate and Breast Cancer Cell Spheroid Cultures Using Gelatin Methacryloyl-Based Hydrogels, Methods in Molecular Biology, 2018, 1786, 175-194.
6. Nair et al., The Thiol-Michael Addition Click Reaction: A Powerful and Widely Used Tool in Materials Chemistry, Chem. Mater., 2014, 26, 1, 724-744.
7. Van Den Bulcke et al, Structural and rheological properties of methacrylamide modified gelatin hydrogels, Biomacromolecules, 2000, 1, 1, 31-38.

1-32. (canceled)

33. A crosslinked modified gelatin, which is produced by reacting a modified gelatin in a click chemical reaction with a crosslinker comprising two or more further reactive moieties, the reactive moieties being reactive in the click chemical reaction with the crosslinkable group reactive moiety incorporated into the modified gelatin,
wherein the modified gelatin is derived from a marine source, and is modified to incorporate a crosslinkable group comprising a thiol moiety, the thiol moiety being reactive in a click chemical reaction, and
wherein the crosslinker further reactive moieties are selected from the group consisting of maleimide, vinyl sulfone, acrylate, acrylamide and methacrylate.

34. A crosslinked modified gelatin according to claim 33, wherein the cross-linker has the formula: 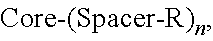
wherein Core is an atom or group providing an attachment for n spacer-R groups, Spacer is a spacer group, R is a group comprising a further reactive moiety, and n is an integer of from 2 to 8.

35. A crosslinked modified gelatin as claimed in claim 34, wherein one or more of the following applies:
(a) Core is C(—CH₂O—)₄; n is 4; Spacer is (—CH₂CH₂O—)ₘ, wherein m is an integer of from 2 to 150; and R is 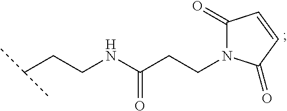
(b) Core is C(—CH₂O—)₄; n is 4; Spacer is (—CH₂CH₂O—)ₘ, wherein m is an integer of from 2 to 50; and R is or 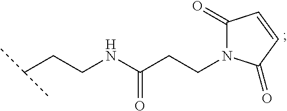
(c) Core is C(—CH₂O—)₄; n is 4; Spacer is (—CH₂CH₂O—)ₘ, wherein m is an integer of from 2 to 150; and R is

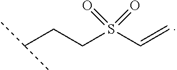

36. A process for producing a crosslinked modified gelatin according to claim 33, comprising reacting a modified gelatin as defined in claim 33, in a click chemical reaction with a cross-linker comprising two or more further reactive moieties as defined in claim 33, the reactive moieties being reactive in the click chemical reaction with the crosslinkable group reactive moiety incorporated into the modified gelatin.

37. A crosslinked modified gelatin produced or producible by a process according to claim 36.

38. A hydrogel comprising a cross-linked modified gelatin according to claim 33, and water.

39. A hydrogel as claimed in claim 38, which is characterizable by one or more of the following:
a) wherein the hydrogel of 2.5% wt/v or greater has a relaxed mass swelling ratio of 15 or less;
b) wherein the hydrogel of between 2.5% and 10% wt/v has an equilibrium swelling ratio of from 10 to 25;
c) wherein the hydrogel of between 2.5% and 10% wt/v has an equilibrium water content of from 80% to 100%;
d) wherein during preparation, the hydrogel of between 2.5% and 10% wt/v has a crosslinking time, at room temperature, of from 1 second to 10 seconds; and
e) wherein during preparation, the hydrogel of between 2.5% and 10% wt/v has a crosslinking time, at room temperature, of from 10 minutes to 30 minutes.

40. A method of making a hydrogel, comprising admixing a crosslinked modified gelatin according to claim 33, and water.

41. A hydrogel which is produced or producible by a method or process according to claim 40.

42. A method of making a hydrogel, comprising carrying out a process of making a crosslinked modified gelatin as claimed in claim 36, in the presence of water.

43. A modified gelatin, wherein the gelatin is derived from a marine source, and is modified to incorporate a crosslinkable group comprising a thiol reactive moiety, the thiol reactive moiety being reactive in a click chemical reaction,
wherein the gelatin has been modified by a reaction selected from the group consisting of:
i) reaction of a gelatin amine group with Traut's reagent;
ii) reaction of a gelatin carboxylic acid group with a diamine, and subsequent reaction with Traut's reagent; and
iii) amide coupling of a gelatin carboxylic acid or carboxylate with an additional cysteine.

44. A modified gelatin according to claim 43, wherein
(a) the number of proline residues is not more than 20% of the total number of amino acid residues in the modified gelatin, or
(b) the number of hydroxyproline residues is not more than 20% of the total number of amino acid residues in the modified gelatin.

45. A modified gelatin according to claim 43, which is characterizable by one or more of the following:
a) wherein the reactive moiety content of the modified gelatin is from about 25 μmol/g to about 1,000 μmol/g;
b) wherein the reactive moiety content of the modified gelatin is from about 300 μmol/g to about 700 μmol/g;
c) wherein an aqueous solution of the modified gelatin of 20% wt/v or lower has a viscosity of below 100,000 mPa·s across a shear rate (l/s) of from 10 to 1,000; and
d) wherein an aqueous solution of the modified gelatin of 20% wt/v or lower has a complex viscosity of below 10⁸ mPa·s across a temperature range of from 0° C. to 40° C.

46. A modified gelatin according to claim 43, wherein the gelatin is a cold-water adapted fish gelatin.

47. A modified gelatin according to claim 46, wherein the cold-water adapted fish is from a genus selected from the group consisting of Salmo, Gadus, Oncorhynchus and Merluccius, preferably Salmo or Oncorhynchus.

48. A process for producing a modified gelatin as defined in claim 43, comprising reacting a gelatin derived from a marine source, with a crosslinkable group precursor by:
i) reaction of a gelatin amine group with a crosslinkable group precursor which is Traut's reagent;
ii) reaction of a gelatin carboxylic acid group with a diamine, and subsequent reaction with Traut's reagent; or
iii) amide coupling of a gelatin carboxylic acid or carboxylate with a crosslinkable group precursor which is cysteine.

49. A modified gelatin which is produced or producible by a process according to claim 48.

50. Use of a hydrogel according to claim 38, as a matrix for cell growth, or for 3D bioprinting.

51. A method of growing cells, comprising:
providing a cell growth matrix comprising a hydrogel according to claim 38; and
growing cells in and/or on the cell growth matrix.

52. A kit for producing a cross-linked modified gelatin comprising:
a) a modified gelatin as claimed in claim 42; and
b) a crosslinker comprising two or more further reactive moieties, the reactive moieties being reactive in the click chemical reaction with the crosslinkable group reactive moiety incorporated into the modified gelatin, wherein the crosslinker further reactive moieties are selected from the group consisting of maleimide, vinyl sulfone, acrylate, acrylamide and methacrylate.
